(19) United States
(12) Patent Application Publication
(10) Pub. No.: US 2010/0092435 A1
(43) Pub. Date: Apr. 15, 2010

(54) USE OF A VARICELLOVIRUS TAP-INHIBITOR FOR THE INDUCTION OF TUMOR-OR VIRUS-SPECIFIC IMMUNITY AGAINST TEIPP

(76) Inventors: Emmanuel Jacques Henri Joseph Wiertz, Utrecht (NL); Danijela Koppers-Lalic, Leiden (NL); Elsa Afra Julia Maria Goulmy, Oegstgeest (NL); Rienk Offringa, Leiden (NL); Thorbald Van Hall, Alphen a/d Rijn (NL)

Correspondence Address: BROWDY AND NEIMARK, P.L.L.C.;624 NINTH STREET, NW, SUITE 300, WASHINGTON, DC, 20001-5303, US

(21) Appl. No.: 12/518,135

(22) PCT Filed: Dec. 7, 2007
(86) PCT No.: PCT/NL2007/050628

§ 371 Date: Oct. 16, 2009

(30) Foreign Application Priority Data
Dec. 7, 2006 (EP) 06125623.6

(51) Int. Cl. A61K 45/00; C12N 5/071; C07H 21/00; C07K 14/00; A61P 37/04

(52) U.S. Cl. 424/93.7; 435/371; 536/23.1; 530/324

(57) ABSTRACT

The present invention provides a novel approach to the modulation of the immune response, directing it towards specific antigens, away from antigens against which no response is desired. The invention is based on the use of viral immune evasion proteins, such as UL49.5, which block antigen presentation to CD8+ T cells. The viral immune evasion proteins are used for: 1) the induction of tumor-specific or virus-specific immunity in cases where a conventional immune response is absent due to antigen processing defects; 2) the induction of empty MHC class I molecules at the cell surface that can be loaded with peptides of a desired specificity; 3) the inhibition of unwanted immune responses against transplanted tissues or organs, e.g. against islets of Langerhans in type 1 diabetes or allogeneic stem cells, or against self antigens in the case of autoimmunity.

### FIELD OF THE INVENTION

The present invention relates to methods wherein varicellovirus TAP-inhibitors are used for induction of tumor- or virus-specific immunity against T cell epitopes associated with impaired peptide processing, and to compositions for use in such methods.

### BACKGROUND OF THE INVENTION

Cytotoxic T lymphocytes (CTL) are important for the immune control of viral infections and have also shown to exhibit the capacity to eradicate established tumors (1-4). The efficacy and safety of CTL-based immunotherapy are currently being evaluated in experimental clinical trials (5-7). An important complication in this respect is the finding that viruses and tumors display diverse mechanisms by which they can evade CTL responses. Especially viruses that cause lifelong persistence in the host, such as the herpesviruses EBV, CMV, VZV and HSV have developed sophisticated immune evasion strategies (8, 9). Reactivation of these viruses is a clinical problem in immune-compromised patients, illustrating the delicate balance between viral persistence and elimination by the CTL immune system. Impairment of antigen presentation via MHC class I, which renders these cells resistant to killing by effector CTL, is also frequently found in tumors (10-13). In general, these impairments are more frequently observed in advanced stages and metastases than in early-stage cancer lesions, suggesting that natural CTL immunity imposes a selective pressure on progression of tumor immune escape variants (14, 15).

One effective mechanism of MHC class I down-modulation is the impediment of the function of the transporter associated with antigen processing (TAP), which mediates the delivery of intracellular peptides for binding to MHC class I molecules in the ER (16). Defects in TAP expression is observed in cancers of diverse origin, including breast, lung, colon as well as cervical carcinomas and melanomas (11, 13, 17). Interestingly, dedicated viral proteins that target the peptide transport process have been demonstrated in CMV (US6), HSV (ICP47), γ herpesvirus 68 (mK3) and varicelloviruses (UL49.5) (18-23).

Recently, we identified the existence of a unique category of CTL that selectively eradicates cells with MHC class I processing defects, like TAP-deficiency (24). Normal cells with an intact antigen processing machinery were not recognized. These CTL detect a novel repertoire of peptide antigens that emerges on the surface due to TAP-, tapasin- or proteasome impairments. Although the peptides are derived from widely distributed self proteins, they are not presented by normal processing-proficient cells, and therefore the immune system considers them as immunogenic neo-antigens (24). We refer to this set of peptides as TEIPP, which stands for ‘T cell Epitopes associated with Impaired Peptide Processing’.

WO 98/25645 discloses practical applications of the concept that prevention of TAP-function leads to recognition of novel, endogenous MHC class I dependent antigens by host T-cells that are not recognized in the presence of a fully functional TAP-molecule. WO 98/25645 discloses that immunization with TAP-deficient cells elicits T-cells directed against epitopes expressed preferentially by TAP-deficient cells and that induction of such T-cells can prevent growth of several tumor targets. In particular, WO 98/25645 suggest the use of autologous cells, especially dendritic cells, that have been treated to express MHC class I dependent epitopes associated with impaired cellular peptide processing and to inject these cells into a patient in order to stimulate T cells to react on these epitopes as presented by tumor cells or virally infected cells. For treating the cells to express epitopes associated with impaired cellular peptide processing WO 98/25645 suggest to use a variety of substances that include viral TAP-inhibitors such as e.g. ICP47 or IE 12, proteasome inhibitors such as the peptide aldehyde Z-Leu-Leu-Leu-H (Peptide Internationals Inc., Louisville, Ky.) or Lactacystin (Calbiochem, La Jolla, Calif.), genes encoding inhibitors of components that take part in the peptide processing of the cell, and substances that inhibit the expression of cellular components that take part in the peptide processing, such as e.g. antisense oligonucleotides or ribozymes.

However, the substances suggested in WO 98/25645 for treating the cells to express epitopes associated with impaired cellular peptide processing have not yet shown satisfactory efficacy. Moreover, the viral TAP-inhibitors suggested in WO 98/25645 are ineffective in murine cells, which complicates for preclinical testing of TEIPP-directed CTL in mice. There is thus a need for improved methods and compositions for treating cells with TAP-inhibitors so that they may be used for eliciting TEIPP-directed CTL in the treatment of cancer and/or infectious disease.

### DESCRIPTION OF THE INVENTION

### Definitions

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species. When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed above. Preferably the region of identity is greater than about 5 bp, more preferably the region of identity is greater than 10 bp.

The term “autologous” is used herein to refer to proteins, nucleic acids, cells, tissues or organs that are obtained from one subject or patient and that are, preferably after some form of ex vivo treatment, returned to, administered to or reimplanted or reinfused into the same subject or patient.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated. A “tissue specific” promoter is only active in specific types of tissues or cells.

As used herein, the term “operably linked” refers to two or more nucleic acid or amino acid sequence elements that are physically linked in such a way that they are in a functional relationship with each other. For instance, a promoter is operably linked to a coding sequence if the promoter is able to initiate or otherwise control/regulate the transcription and/or expression of a coding sequence, in which case the coding sequence should be understood as being “under the control of” the promoter. Generally, when two nucleic acid sequences are operably linked, they will be in the same orientation and usually also in the same reading frame. They will usually also be essentially contiguous, although this may not be required.

The terms “signal sequence”, “signal peptide” and “secretory leader” are used interchangeably and refer to a short (usually about 15-60 amino acids), continuous stretch of amino acids usually present at the amino-terminus of secreted and membrane-bound polypeptides and that directs their delivery to various locations outside the cytosol. Thus, specific sorting or targeting signals, which include signal sequences, may direct the delivery of polypeptides into the nucleus, ER, mitochondria, peroxisomes, etc. Signal sequences usually contain a hydrophobic core of about 4-15 amino acids, which is often immediately preceded by a basic amino acid. At the carboxyl-terminal end of the signal peptide there are a pair of small, uncharged amino acids separated by a single intervening amino acid that defines the signal peptide cleavage site. von Heijne, G. (1990) J. Membrane Biol. 115: 195-201. Despite their overall structural and functional similarities, native signal peptides do not have a consensus sequence.

A “transgene” is herein defined as a gene that has been newly introduced into a cell, i.e. a gene that does not normally occur in the cell. The transgene may comprise sequences that are native to the cell, sequences that in naturally do not occur in the cell and it may comprise combinations of both. A transgene may contain sequences coding for one or more proteins that may be operably linked to appropriate regulatory sequences for expression of the coding sequences in the cell.

For purposes of the present invention, the degree of identity, i.e. the match percentage, between two polypeptides, respectively two nucleic acid sequences is preferably determined using the optimal global alignment method CDA (Huang, 1994, A Context Dependent Method for Comparing Sequences, Proceedings of the 5th Symposium on Combinatorial Pattern Matching, Lecture Notes in Computer Science 807, Springer-Verlag, 54-63) with the parameters set as follows: (i) for (poly)peptide alignments: Mismatch: −2 GapOpen: 11 GapExtend: 1 ContextLength: 10 MatchBonus: 1, and (ii) for nucleotide sequence alignments Mismatch: −15 GapOpen: 5 GapExtend: 2 ContextLength: 10 MatchBonus: 1. The terms “degree of identity”, “identity” and “percentage of homology” are used interchangeably to indicate the degree of identity between two polypeptides or nucleic acid sequences as calculated by the optimal global alignment method indicated above. Examples of alternative programs used for alignments and determination of homology are Clustal method (Higgins, 1989, CABIOS 5: 151-153), the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.), BLAST (NCBI), GAP (Huang) for the optimal global alignments, MAP (Huang), MultiBLAST (NCBI), ClustalW, Cap Assembler and Smith Waterman for multiple alignments. Software for performing BLAST analyses is publicly available through the National Center for Biotechnology Information (http://www.ncbi.nlm.nih.gov/).

In a first aspect the invention relates to a method for producing a cell that is capable of activating CD8⁺ T cells that selectively recognize cells presenting TEIPP, the method comprising treating the cell with a source of a varicellovirus TAP-inhibitor.

A varicellovirus TAP-inhibitor according to the present invention is a protein that preferably reduces TAP-dependent peptide transport in a mammalian cell by at least 50, 60, 70, 80, 90, or 95%. More preferably the TAP-inhibitor reduces TAP-dependent peptide transport in human and/or murine cells. Suitable assays for inhibition of TAP-dependent peptide transport are described in Example 2.1.3 herein for both human (2.1.3.1) and mouse (2.1.3.2) cells. A particularly preferred TAP inhibitor according to the present invention reduces TAP-dependent transport of the fluorescein-conjugated synthetic peptide CVNKTERAY in cells of the human melanoma MEL-JUSO (MJS) cell line that stably express the TAP-inhibitor by at least 50, 60, 70, 80, 90, or 95%, as compared to TAP-dependent transport of the peptide in untransformed human melanoma (MJS) cells, under the same conditions. The human melanoma MEL-JUSO cell line is available from DSMZ, Braunschweig, Germany (www.dsmz.de) under accession no. ACC 74.

A varicellovirus TAP-inhibitor according to the present invention may further be a protein that has at least 50, 60, 70, 80, 90, or 95% amino acid identity with at least one of SEQ ID NO.'s 1, 2, 3 and 4. A preferred varicellovirus TAP-inhibitor according to the present invention is a protein that an amino acid sequence as depicted in SEQ ID NO.'s 1, 2, 3 or 4, of which SEQ ID NO.'s 2, 3 and 4 are more preferred, SEQ ID NO.'s 3 and 4 are still more preferred and SEQ ID NO. 1 is most preferred. The inventors have found that a varicellovirus TAP-inhibitor with the amino acid sequence of SEQ ID NO. 1, the BHV-1 UL49.5 protein, shows a reduction of peptide transport by 98%; a varicellovirus TAP-inhibitor with the amino acid sequence of SEQ ID NO. 2, the PRV UL49.5 protein, shows a reduction of peptide transport by 78%; a varicellovirus TAP-inhibitor with the amino acid sequence of SEQ ID NO. 2, a varicellovirus TAP-inhibitor with the amino acid sequence of SEQ ID NO. 3, the EHV-1 UL49.5 protein, shows a reduction of peptide transport by 95%.

A further preferred varicellovirus TAP-inhibitor according to the present invention is a varicellovirus TAP-inhibitor as defined above, wherein the TAP-inhibitor is modified to improve its stability. A TAP-inhibitor with improved stability is herein understood as a TAP-inhibitor that has an increased half-life in human and/or murine cells, preferably in human melanoma (MJS) cells, as compared to the corresponding unmodified (i.e. wild type) TAP-inhibitor. Increases in half-life of modified TAP-inhibitors of the invention may be determined in human melanoma (MJS) cells as described in Example 4 herein. A modified TAP-inhibitors of the invention with improved stability preferably is a TAP-inhibitor with one or more modifications in the cytoplasmic tail of the TAP-inhibitor. The cytoplasmic tail of the TAP-inhibitor is herein defined as the amino acid sequence that is C-terminal to the transmembrane domain of the TAP-inhibitor: e.g. amino acids 59-75 in SEQ ID NO. 1, amino acids 58-73 in SEQ ID NO. 2, amino acids 58-73 in SEQ ID NO. 3, amino acids 59-74 in SEQ ID NO. 4, or corresponding amino acids in other TAP-inhibitors. The modification in the cytoplasmic tail of the varicellovirus TAP-inhibitor preferably is a modification that prevents or reduces ubiquitination of the TAP-inhibitor. Preferably therefore, in the modification of the TAP-inhibitor at least one of the lysine, serine, threonine and cysteine residues in the cytoplasmic tail of the TAP-inhibitor is deleted or replaced with an amino acid residue other than lysine, serine, threonine and cysteine. Preferably, the cytoplasmic tail of the modified TAP-inhibitor at least lacks lysine residues and/or the tail at least lacks serine and threonine residues, and/or the tail lacks cysteine residues. More preferably the tail lacks lysine, serine, and threonine residues. Most preferably the tail lacks lysine, serine, threonine and cysteine residues. In modifying the cytoplasmic tail of a TAP-inhibitor, replacement of a lysine, serine, threonine or cysteine residue with an amino acid other than lysine, serine, threonine and cysteine is preferred over its deletion. More preferably the replacement is a conservative replacement such as e.g. replacing each serine, threonine or cysteine with alanine and replacing lysine with arginine. One example of a modified TAP-inhibitor with improved stability is the modified BHV-1 UL49.5 protein described in Example 4 wherein the lysine residues at positions 68 and 69 (see SEQ ID NO. 1) have been replaced with alanine residues.

In accordance with the invention, the source of a varicellovirus TAP-inhibitor may be any composition that may administered to the cells and that, when administered in an effective dose, is capable of effecting a functional level of varicellovirus TAP-inhibitor in the cell. A functional level of TAP-inhibitor in the cell is understood to mean a level that reduce TAP dependent peptide transport in the cell by at least 40, 50, 60, 70, or 80%. The source of a varicellovirus TAP-inhibitor may thus be a composition comprising the TAP-inhibitor protein. Such a TAP-inhibitor protein composition may be any formulation that is suitable for introducing the protein into the cell, e.g. by means of microinjection or electroporation, or the TAP-inhibitor protein may be packaged in liposomes to facilitate its introduction into the cell.

A preferred source of a varicellovirus TAP-inhibitor is however a nucleic acid molecule encoding the TAP-inhibitor. A nucleic acid molecule encoding the TAP-inhibitor may be a DNA molecule or it may be an RNA molecule. Preferably the nucleic acid molecule encoding the TAP-inhibitor is an expression construct. The expression construct can be any nucleic acid construct comprising a nucleotide sequence encoding a varicellovirus TAP-inhibitor that is suitable for introduction into the desired target cell and that is capable of expressing the TAP-inhibitor upon introduction into the cell. In the expression construct, the nucleotide sequence encoding the mature TAP-inhibitor is preferably operably linked to expression signals, including e.g. translation initiation sequences, a signal sequence and/or transcription regulatory sequences such as e.g. a promoter. The expression signals preferably allow expression of a nucleotide sequence encoding TAP-inhibitor in the target cell. Thus, in the expression construct the nucleotide sequence encoding the mature TAP-inhibitor is preferably operably linked to a nucleotide sequence encoding a signal sequence to direct translocation of the TAP-inhibitor into the ER of the cells expressing the construct. Preferably, the sequence encodes a signal sequence that is native to the sequence encoding the mature TAP-inhibitor, e.g. the signal sequence consisting of amino acid 1-21 of SEQ ID NO. 5 (BHV1), amino acids 1-25 of SEQ ID NO. 6 (PRV), amino acids 1-27 of SEQ ID NO. 7 (EHV1), or amino acids 1-26 of SEQ ID NO. 8 (EHV4). However, other suitable signal sequence that are capable of directing translocation of the TAP-inhibitor into the ER of the cell may also be applied.

In the expression construct the nucleotide sequence encoding varicellovirus TAP-inhibitor preferably is operably linked to a promoter. The promoter is a promoter that is preferably active or can be induced to be active in the mammalian target cell, preferably an antigen presenting cell, such as a dendritic cell. The promoter may be a constitutive promoter, an inducible promoter or a tissue specific promoter, preferably specific for an antigen presenting cell, such as a dendritic cell. Suitable promoters for expression of the nucleotide sequence encoding an TAP-inhibitor include e.g. cytomegalovirus (CMV) intermediate early promoter, viral long terminal repeat promoters (LTRs), such as those from murine moloney leukaemia virus (MMLV), rous sarcoma virus, or HTLV-1, the simian virus 40 (SV 40) early promoter and the herpes simplex virus thymidine kinase promoter, the human IL-2 promoter and the DC-specific CD11c promoter. The expression construct may further comprise additional sequence elements for the expression of the nucleotide sequence encoding an TAP-inhibitor, such as transcriptional enhancers and/or silencers, transcriptional terminators, and polyA-addition sites.

The expression construct may optionally comprise a second or one or more further nucleotide sequence coding for a second or further protein. The second or further protein may be a (selectable) marker protein that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York.

Alternatively, the second or further nucleotide sequence may encode a protein that provides for fail-safe mechanism that allows to cure a subject from the TAP-inhibitor transgenic cells of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the IL-10 transgenic cells in the subject (see e.g. Clair et al., 1987, Antimicrob. Agents Chemother. 31: 844-849). The nucleotide sequence coding for the marker protein is preferably also operably linked to a promoter for expression in the mammalian target cell (e.g. an antigen presenting cell, such as a dendritic cell) as described above for the nucleotide sequence encoding an TAP-inhibitor.

The expression construct may be in the form of any nucleic acid capable of being introduced into the mammalian target cell. The expression construct may be DNA, RNA or a combination of both; it may be a naked nucleic acid molecule, such as a plasmid or a linear DNA or RNA fragment; and it may be a single or a double stranded nucleic acid molecule. The expression construct may thus be a non-viral vector such as a plasmid or linear nucleic acid that may be packaged in e.g. a liposome for efficient delivery into the mammalian target cell. Alternatively, the expression construct is a viral vector that may be used to transduce or infect the mammalian target cell. The expression construct preferably is safe, efficient, and reliable and allows for expression, preferably controlled expression of the TAP-inhibitor transgene, and for some therapeutic purposes long term expression of the transgene is preferred. The construct may e.g. be a viral vector which are more efficient agents for gene transfer as compared to the non-viral agents. Suitable viral expression constructs include e.g. vectors that are based on adenovirus, adeno-associated virus (AAV) or retroviruses as recently reviewed (42, 43, 44). Preferred retroviral expression constructs for use in the present invention are lentiviral based expression constructs. Lentiviral vectors have the unique ability to infect non-dividing cells. Methods for the construction and use of lentiviral based expression constructs are described in U.S. Pat. Nos. 6,165,782, 6,207,455, 6,218,181, 6,277,633 and 6,323,031.

However, in a preferred embodiment the nucleic acid molecule encoding a varicellovirus TAP-inhibitor for use in the present invention is a molecule that allows only transient expression of the TAP-inhibitor. The nucleic acid molecule is thus a molecule that does not stably transfect or transform the cell. The nucleic acid molecule, therefore preferably is an expression construct that does not integrate into the host cell's genome, e.g. the construct is a non-integrating construct, an episomal construct. Such a construct integrates only with very low efficiency (preferably less than 10⁻³, 10⁻⁴, 10⁻⁵, or 10⁻⁶ of all transduced cells). A construct that lacks recombinogenic ends, such as a circular construct is therefore preferred. For transient expression the nucleic acid molecule preferably also is a non-replicating construct, e.g. does not comprise an origin of replication that functions in the mammalian target host cell.

A particularly preferred nucleic acid molecule encoding a varicellovirus TAP-inhibitor for transient expression thereof is a RNA molecule encoding the TAP-inhibitor, which RNA molecule upon introduction into the cell is capable of being translated to produce TAP-inhibitor protein in the mammalian target cell. Suitable RNA molecules encoding the TAP-inhibitor and that are capable of being translated upon introduction into the mammalian target cell may be obtained by in vitro transcription using e.g. a T7 polymerase in vitro transcription vector (e.g. pGEM4Z; Promega), comprising the TAP-inhibitor coding sequence. RNA may be transcribed in vitro using T7 RNA polymerase and a cap analogue, as described previously (45; Ambion mMessage mMachine kit). A suitable a cap analogue is e.g. 5′ 7-methyl guanosine nucleotide (m7G(5′)ppp(5′)G; Ribo m7G Cap Analog as obtainable from Promega). In a preferred composition comprising RNA molecules encoding the TAP-inhibitor at least 50, 60, 70, 80 or 90% of the RNA molecules comprise a cap or cap analog. The in vitro transcribed RNA molecule encoding the TAP-inhibitor may be electroporated into the mammalian target cell, preferably an antigen presenting cell, such as a dendritic cell as described (46, 47).

Preferably, in a nucleic acid molecule encoding a varicellovirus TAP-inhibitor for use in the present invention, the TAP-inhibitor coding sequence is adapted for improved expression in the mammalian target cell. The nucleotide sequence encoding the TAP-inhibitor may be adapted to optimize its codon usage to that of the mammalian, preferably human, target host cell. The adaptiveness of a nucleotide sequence encoding the TAP-inhibitor to the codon usage of the host cell may be expressed as codon adaptation index (CAI). The host cell to which the codon usage is adapted preferably is a human cell, more preferably a hematopoietic cell. The codon adaptation index is herein defined as a measurement of the relative adaptiveness of the codon usage of a gene towards the codon usage of highly expressed genes. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Kim et al., Gene. 1997, 199:293-301; zur Megede et al., Journal of Virology, 2000, 74: 2628-2635). An adapted nucleotide sequence preferably has a CAI of at least 0.3, 0.4, 0.5, 0.6, 0.7, 0.8, or 0.9. A preferred nucleotide sequence encoding a varicellovirus TAP-inhibitor wherein at least 50, 75, 90, 95, 98 or 99%, and preferably all of the non-common codons or less-common codons are replaced with a common codon encoding the same amino acid as outlined in Table 3. A common codon is herein meant the most common codon encoding each particular amino acid residue in highly expressed human genes as shown in Table 3. Common codons thus include: Ala (gcc); Arg (cgc); Asn (aac); Asp (gac); Cys (tgc); Gln (cag); Gly (ggc); H is (cac); Ile (atc); Leu (ctg); Lys (aag); Pro (ccc); Phe (ttc); Ser (age); Thr (acc); Tyr (tac); Glu (gag); and Val (gtg) (see Table 1). “Less-common codons” are codons that occurs frequently in humans but are not the common codon: Gly (ggg); Ile (att); Leu (etc); Ser (tcc); Val (gtc); and Arg (agg). All codons other than common codons and less-common codons are “non-common codons”. Preferably the nucleotide sequence encoding a varicellovirus TAP-inhibitor has a continuous stretch of at least 25, 50, 60, or 75 codons all of which are common codons. The TAP-inhibitor coding sequence may further be adapted for improved expression in the mammalian target cell by methods described in WO 2004/059556, and by modifying the CpG content of the coding sequence as described in WO 2006/015789. A particularly preferred TAP-inhibitor coding sequence that is adapted for improved expression in the mammalian target cell is the TAP-inhibitor coding sequence of SEQ ID NO. 10. A preferred nucleotide sequence encoding the TAP-inhibitor is therefore a nucleotide sequence encoding the amino acid sequence of SEQ ID NO. 1 or 5, and that has at least 60, 70, 80, 90, or 95% identity with SEQ ID NO. 10.

In a preferred embodiment of the methods of the invention, the treatment of the cell with the source of a varicellovirus TAP-inhibitor is combined with treating the cell with at least one of a source of an ICP47 (derived from Herpes Simplex Virus) TAP-inhibitor and a source of a US6 (derived from Cytomegalo Virus) TAP-inhibitor.

An ICP47 TAP inhibitor according to the present invention reduces TAP-dependent transport of the fluorescein-conjugated synthetic peptide CVNKTERAY in cells of the human melanoma MEL-JUSO (MJS) cell line that stably express the TAP-inhibitor by at least 50, 60, 70, 80, 90, or 95%, as compared to TAP-dependent transport of the peptide in untransformed human melanoma (MJS) cells, under the same conditions. An ICP47 TAP-inhibitor according to the present invention may further be a protein that has at least 50, 60, 70, 80, 90, or 95% amino acid identity with at least one of SEQ ID NO.'s 11 and 12.

A US6 TAP inhibitor according to the present invention reduces TAP-dependent transport of the fluorescein-conjugated synthetic peptide CVNKTERAY in cells of the human melanoma MEL-JUSO (MJS) cell line that stably express the TAP-inhibitor by at least 50, 60, 70, 80, 90, or 95%, as compared to TAP-dependent transport of the peptide in untransformed human melanoma (MJS) cells, under the same conditions. A US6 TAP-inhibitor according to the present invention may further be a protein that has at least 50, 60, 70, 80, 90, or 95% amino acid identity with at least one of SEQ ID NO.'s 13.

The sources of ICP47 and US6 TAP-inhibitor may be any composition that may be administered to the cells and that, when administered in an effective dose, is capable of effecting a functional level of varicellovirus TAP-inhibitor in the cell. A functional level of TAP-inhibitor in the cell is understood to mean a level that reduce TAP dependent peptide transport in the cell by at least 40, 50, 60, 70, or 80%. The source of ICP47 and/or US6 TAP-inhibitor may thus be a composition comprising the ICP47 and/or US6 TAP-inhibitor protein as described above for source of a varicellovirus TAP-inhibitor may thus be. A preferred source of ICP47 and/or US6 TAP-inhibitor is however a nucleic acid molecule encoding the TAP-inhibitor(s) as described or defined above for the source of a varicellovirus TAP-inhibitor. All three herpes viral TAP-inhibiting proteins ICP47 (derived from Herpes Simplex Virus), US6 (derived from Cytomegalo Virus) and UL49.5 (derived from Varicello Virus) act with distinct mechanisms: ICP47 blocks the pore, US6 prevents the energy supply and UL49.5 directs the breakdown of TAP and, as a consequence, the combination of these inhibitors should result in highly efficient blockage of the peptide transport. The combination of one or more of these inhibitors is synergistic. Therefore lower amounts of the individual TAP inhibitors may be used when they are applied in combination. E.g. when applied in combination the dosage of each individual TAP inhibitor in the combination is at least the amount that the reduces TAP dependent peptide transport in the cell by at least 30, 40, 50, 60, or 70% when the individual TAP inhibitor is applied alone.

The method of the invention for producing a cell that is capable of activating CD8⁺ T cells that selectively recognize cells presenting TEIPP, preferably is a method wherein the cell is treated in vitro or ex vivo with a source of a varicellovirus TAP-inhibitor, i.e. the method preferably is an in vitro method. The cell that is treated with a source of a varicellovirus TAP-inhibitor upon (re-)introduction activates CD8⁺ T cells that selectively recognize cells presenting TEIPP, in other words, the cell elicits, induces or arouses a TEIPP-specific CTL response in a system capable of exhibiting said response. The TEIPP-specific CTL response preferably is a MHC class I dependent TEIPP-specific CTL response. The system capable of exhibiting said response may be an in vitro system but preferably is a human or animal subject in need of a TEIPP-specific CTL response. The human or animal in need of a TEIPP-specific CTL response may be a subject comprising tumor cells and/or virally infected cells that present T cell Epitopes associated with Impaired Peptide Processing (TEIPP).

The mammalian target cell that is treated with a source of a varicellovirus TAP-inhibitor preferably is a human or a murine cell. The target cell preferably is a hematopoietic cell, such as e.g. lymphocytes, B cells, T cells, CD4+ cells, monocytes or dendritic cells (DC), MHC class II-positive or -negative cells, or combinations of these cells. Specific subfractions of such hematopoetic cell may be enriched from peripheral blood mononuclear cells (PBMC), including e.g. lymphocytes, B cells, T cells, CD4+ cells, monocytes or dendritic cells (DC), MHC class II-positive or -negative cells, or combinations of these cells. Various methods are available in the art for the enrichment for specific subfractions present in the PBMC bulk. Specific subfractions of PBMCs may e.g. be enriched by red cell lysis, density centrifugation, by sorting on cell-sorter using fluorescent labeling of cell surface markers specific for a given subset of PBMCs, or by expanding specific subsets of PBMCs by incubation of the PBMCs under conditions that favor the proliferation and development of a given subset of PBMCs, e.g. using specific growth factors and/or interleukins (see e.g. 50).

A preferred mammalian target cell that is treated with a source of a varicellovirus TAP-inhibitor preferably is an antigen presenting cell, such as e.g. a dendritic cell. The antigen presenting cell preferably is a cell capable of activating CD8⁺ T cells. More preferably, the antigen presenting cell is a dendritic cell that is matured, as defined by high expression of CD80 (=B7.1), CD86 (=B7.2), CD40 and MHC class I and MHC class II molecules.

Antigen presenting cells, such as dendritic cells, can be enriched or isolated from peripheral blood by methods known in the art per se. They can e.g. be sorted from peripheral blood (PBMC) by immunomagnetic sorting to molecules such as CD34 or CD 14. Magnetic beads can be obtained from Dynal. They can be grown in vitro in suitable medium, e.g. IMDM (Life Technologies, Inc., Grand Island, N.Y.) with appropriate supplements (48) and various adjuvants to improve development and immunogenicity. Examples of adjuvants are cytokines such as Granulocyte-Macrophage colony stimulating factor (GM-CSF), IL-4, Tumor Necrosis Factor α (TNF-α), stem cell factor (SCF) or Transforming Growth Factor—β(TGF-β), antibodies to MHC Class II or CD40 (which enhance B7 expression) or genes for costimulatory molecules.

Another preferred mammalian target cell that is treated with a source of a varicellovirus TAP-inhibitor preferably is a B cell as may be enriched from PBMC as indicated (50).

The mammalian target cell preferably is an autologous cell. The autologous cell is preferably obtained from human or animal subject in need of a TEIPP-specific CTL response. The mammalian target cell preferably is a primary cell as opposed to a cell line. The cell therefore is a mortal cell (i.e. not immortalized) that is not tumorigenic and/or transformed.

Thus, in a further aspect the invention relates to a cell that has been treated with a source of a varicellovirus TAP-inhibitor in a method as defined above, for use in the treatment of cancer or a virus infection. Preferably, the cancer is a tumor of cells with impaired peptide processing and/or the virus causes impaired peptide processing in cell infected by the virus, such as e.g. herpes viruses like EBV, CMV, VZV and HSV. Preferably the cell is used for activating CD8⁺ T cells that selectively recognize cells presenting TEIPP. Thus the cells of the invention that have been treated to express TEIPP may be used for the manufacture of a pharmaceutical composition or a vaccine against cancer or virus infections and/or to activate CD8⁺ T cells that selectively recognize tumor- or virally infected cells presenting TEIPP, preferably MHC class I dependent TEIPP. A composition of cells of the invention, that have been treated with a source of a varicellovirus TAP-inhibitor in a method as defined above and that are capable of activating CD8⁺ T cells that selectively recognize cells presenting TEIPP, may then be injected into a subject/patient in order to stimulate T cells (CTLs) to react on cells expressing these TEIPP.

Cells, that have been treated with a source of a varicellovirus TAP-inhibitor in a method as defined above, may be used for activation in vivo or in vitro of T cells (CD8⁺) against TEIPP. The in vivo procedure is described above. The in vitro procedure could be e.g. as follows: a) cells are treated with a source of a varicellovirus TAP-inhibitor, as described above b) T cells are isolated (e.g. from PBMC) and stimulated in vitro with the cells obtained in step a; and c) activated T cells are given to the patient. Preferably the activated T cells are autologous to the patient. Stimulation of T-cells in vitro with dendritic cells that have been treated with a source of a varicellovirus TAP-inhibitor, may be done in accordance with to standard procedures, e.g. T-cells are sorted out from peripheral blood and cultured in the presence of dendritic cells in appropriate media and appropriate additives e.g. MEM media and IL-2 (48, 49).

The invention further relates to a pharmaceutical preparation comprising as active ingredient a cell or a source of a varicellovirus TAP-inhibitor as defined above including combinations with sources of other viral TAP-inhibitors as defined above. The composition preferably at least comprises a pharmaceutically acceptable carrier in addition to the active ingredient. The preferred form depends on the intended mode of administration and therapeutic application. The pharmaceutical carrier can be any compatible, non-toxic substance suitable to deliver the polypeptides to the patient. Sterile water, alcohol, fats, waxes, and inert solids may be used as the carrier. Pharmaceutically acceptable adjuvants, buffering agents, dispersing agents, and the like, may also be incorporated into the pharmaceutical compositions.

The cells obtained in any of the methods of the invention are administered parentally. Preparations for parental administration must be sterile and physiologically tolerable. Sterilization is readily accomplished by filtration through sterile filtration membranes, prior to or following lyophilization and reconstitution. The parental route for administration of the cells of the invention is in accord with known methods, e.g. injection or infusion by intravenous, intraperitoneal, intramuscular, intraarterial or intralesional routes. The cells may be administered continuously by infusion or by bolus injection. Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes. A typical composition for intravenous infusion could be made up to contain 10 to 50 ml of sterile 0.9% NaCl or 5% glucose optionally supplemented with a 20% albumin solution and 10² to 10¹⁰ cells. Methods for preparing parenterally administrable compositions are well known in the art and described in more detail in various sources, including, for example, Remington's Pharmaceutical Science (15th ed., Mack Publishing, Easton, Pa., 1980) (incorporated by reference in its entirety for all purposes).

In a further aspect the invention relates to a nucleic acid molecule comprising a nucleotide sequence encoding a varicellovirus TAP-inhibitor as herein defined above, including combinations with nucleotide sequences encoding other viral TAP-inhibitors as defined above, as well as to a composition comprising such a nucleic acid molecule. The compositions may be used in any of the methods of the invention.

In yet another aspect the invention pertains to a modified varicellovirus TAP-inhibitor with improved stability as defined above and to compositions comprising such modified TAP-inhibitors.

In a further aspect the invention pertains to a method for producing a cell that presents an empty MHC class I molecule at its cell surface, the method comprising treating the cell with a source of a varicellovirus TAP-inhibitor. The cell is a mammalian target cell, preferably is a hematopoetic cell, as herein defined above. The cell is treated with a source of a varicellovirus TAP-inhibitor as herein defined above, including combinations with other viral TAP-inhibitors as defined above. The method preferably is an in vitro method. The method is thus used for induction of empty MHC class I molecules at the cell surface that can be loaded with peptides of a desired specificity, e.g. synthetic peptides comprising a MHC class I epitope of a tumor- or microbial-antigen. Cells presenting MHC class I molecules with exogenously added (synthetic) peptides may then be used to induce T cell immunity, e.g. a CTL response, against the tumor- or microbial-antigen in the treatment of cancer or an infectious disease. The invention further relates to cells obtained in this method and compositions comprising those cells.

In a further aspect the invention pertains to a method for producing a cell having reduced surface expression of MHC class I molecules at its cell surface, the method comprising treating the cell with a source of a varicellovirus TAP-inhibitor. Reduced expression of MHC class I molecules is understood to mean a reduction of at least 20, 30, 40, 50, 60, 80 or 90% as compared to a cell that has not been treated with the TAP inhibitor. The cell is treated with a source of a varicellovirus TAP-inhibitor as herein defined above, including combinations with other viral TAP-inhibitors as defined above. The cell is a mammalian target cell, preferably is a human cell of a tissue to be transplanted. The cell to be transplanted is treated with the source of TAP-inhibitors in order to reduce or the inhibit unwanted immune responses against transplanted tissues or organs, e.g. against transplanted (cells of) islets of Langerhans in type 1 diabetes, beta cells, allogeneic stem cells, or against self tissue/self antigens in the case of autoimmunity. Because in this method a long term effect of the reduced immunogenicity is desired, preferably the source of viral TAP-inhibitors preferably is one or more nucleic acid expression constructs for long term expression, such as e.g. lentiviral based expression constructs. The invention further relates to cells obtained in this method and compositions comprising those cells.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### DESCRIPTION OF THE FIGURES

FIG. 1. TEIPP-specific CTL selectively recognize TAP-deficient dendritic cells.

(A) Previously established TEIPP-specific CTL clone (left panel) or RMA tumor-specific CTL clone (right panel) were tested for recognition of dendritic cells (DC) from wild type B6 mice, DC from TAP1−/− mice or RMA lymphoma cells. Dendritic cells were derived from bone marrow as described in material and methods. IFNγ release by CTL was measured after 18 h of co-incubation. Means and standard deviations of triplicate wells are depicted and one out of two comparable experiments is shown.
(B) Two independently derived TEIPP-specific CTL clones were tested (left and right panels) for reactivity against TAP-deficient tumor cells. RMA-S is a TAP2-deficient variant of the RMA lymphoma and MCA is a fibrosarcoma from a TAP1−/− mouse (24). TAP function of MCA was restored by gene transfer (‘MCA.TAP1’). C4.4-25 is a β2m-negative lymphoma and is included to control for non-specific activity by the CTL clones. Means and standard deviations of triplicate wells are depicted and one out of three comparable experiments is shown.

FIG. 2. Immunization with TAP1−/− dendritic cells protects mice for outgrowth of TAP-deficient tumor variants.

(A-B) C57BL/6 mice were injected with syngeneic dendritic cells from wild type mice (‘B6 DC’), dendritic cells from mice with TAP1−/− genetic background (‘TAP1−/− DC’) or saline solution (‘naïve’). Mice were challenged with TAP-deficient RMA-S lymphoma cells (A) or TAP-deficient MCA fibrosarcoma cells (B) and progressive tumor growth was measured. Mice were sacrificed when tumors reached a volume of 1000 mm³. NK cells were depleted during the whole experiment to prevent NK-mediated kill of tumor cells in vivo. The survival curves shown represent two compiled experiments with 10 mice in each group. The differences between groups B6 DC and TAP1−/− DC are statistically significant (log rank test, P=0.004 for A and P<0.0001 for B).
(C) CD8+ cells are responsible for the protection against TAP-deficient tumors. C57BL/6 mice were treated with syngeneic dendritic cells from wild type mice (‘control B6 DC’) or from TAP1−/− mice (‘TAP1−/− DC’). CD8+ or CD4+ cells were depleted by injection of specific mAbs just before tumor challenge with RMA-S cells. Progressive tumor growth was measured and average tumor size per group (n=5) is depicted. NK cells were depleted during the whole experiment to prevent NK-mediated lysis of tumor cells in vivo. Statistical analysis using Log rank test revealed these results ‘TAP1−/− DC’ vs ‘TAP1−/− DC, CD8-depleted’ (P=0.02) and ‘TAP1−/− DC vs ‘TAP1−/− DC, CD4-depleted’ (P=0.004).

FIG. 3. A viral TAP-inhibitor induces the presentation of TEIPP antigens in dendritic cells.

(A) Dendritic D1 cells were retrovirally transduced with the UL49.5 gene of Bovine Herpes Virus-1 (thin lines) or empty vector (thick lines). Cell surface expression of MHC class I (upper panel) and CD40 molecules (lower panel) were detected using specific antibodies. Filled histograms represent background staining without antibodies.
(B) UL49.5-expressing (‘DC.UL49.5’) or control (‘DC.vector) dendritic D1 cells were used as targets for two TEIPP-specific CTL clones. IFNγ release upon co-incubation with TAP-deficient RMA-S cells was comparable for both CTL clones (13 and 17 μg/ml, respectively). Means and standard deviations of triplicate wells are depicted and one out of two comparable experiments is shown.

FIG. 4. Substitution of lysine residues in the cytoplasmic tail of BHV1-UL49.5 increases the stability of the viral inhibitor. MJS cells were transduced with retroviruses to express wild type UL49.5 or a recombinant form in which the two lysine residues in the cytoplasmic tail have been substituted for alanines. To evaluate the stability of these proteins, the cells were pulse-labeled with [35]S-methionine/cysteine and chased for 4 and 8 hrs. UL49.5 wt and UL49.5 KK/AA were immunoprecipitated from cell lysates, separated on SDS-PAGE and displayed using phosphoimaging technology.

### EXAMPLES

### 1. Example 1

### TAP-Inhibiting Proteins US6, ICP47 and UL49.5 Differentially Affect Minor and Major Histocompatibility Antigen-Specific Recognition by Cytotoxic T Lymphocytes

### 1.1 Materials and Methods

### 1.1.1 Retroviral Constructs

cDNA's encoding the viral proteins US6, ICP47 and UL49.5 were generated by PCR under standard conditions. Plasmids containing the US6 and ICP47 genes were kind gifts of Dr. J. Neefjes (Dutch Cancer Institute, Amsterdam) and Dr. K. Frith (Vaccine and Gene Therapy Institute, Oregon Health and Science University), respectively. The PCR-generated products were inserted into the pLZRS-polylinker-IRES-eGFP retroviral vector (http://www.stanford.edu/group/nolan/protocols/pro_helper_free.html) upstream of the internal ribosomal entry site (IRES) and enhanced GFP. Retrovirus production and transduction of EBV-LCL were performed as described (http://www.stanford.edu/group/nolan/protocols/pro_helper_free.html).

### 1.1.2 Cell Lines

EBV-LCLs Modo and Hodo (Table 1) were transduced with retroviral vectors to generate the following stable GFP-positive cell lines: Modo-control and Hodo-control (containing a retroviral vector without insert); Modo-US6; Modo-ICP47; Modo-UL49.5 and Hodo-UL49.5. GFP-positive cells were selected by a FACS Vantage cell sorter (Becton Dickinson, San Jose, Calif.) to ensure homogenous and comparable expression of the various TAP-inhibitors. All EBV-LCLs were cultured in IMDM containing 5% FCS.

TABLE 1
HLA class I and mHag genotyping of the various EBV-LCLs
used in this study.

| EBV- | | | | | | |
|---|---|---|---|---|---|---|
| LCLs | HLA-A | HLA-B | HLA-C | mHags | | |
| Modo | A2 | B44, B60 | C5, C10 | HY | HA-1 | HA-2 |
| Hodo | A1, A11 | B8, B60 | C3, C7 | HY | — | — |
| H6 | A2 | B27, B62 | C1, C3 | — | — | — |
| T2 | A2 | B51 | C2 | — | HA-1 | HA-2 |

In vitro generation of mHag- and alloHLA-specific CTL clones is documented in detail elsewhere (25, 26). Clone #1 was kindly donated by Prof. J. H. F. Falkenburg (Leiden University Medical Center). All CTL clones were cultured in IMDM containing 10% pooled human serum and 25 U/ml interleukin-2 (Cetus, Emeryville, Calif.).

### 1.1.3 Synthetic Peptides and Human Monoclonal Antibodies

HA-1, HA-2 and HY peptides were synthesized according to their reported sequences (27-29). Where stated, EBV-LCLs were pulsed with 10 μg/ml of relevant mHag peptides for 1 hour at 37° C.

Hybridomas producing human monoclonal antibodies (mAbs) SN607D8 (anti HLA-A2/A28), VTM1F11 (anti HLA-B7/B27/B60) and GV5D1 (anti HLA-A1/A9) were generated as described previously [30]. The HLA-specificities of these mAbs (all IgG) were determined by complement-mediated cytotoxicity assays against large (n>240) panels of serologically typed peripheral blood mononuclear cells. The mAbs were purified by protein A chromatography (Pharmacia, Uppsala, Sweden) and biotin-labeled (Pierce, Rockford, Ill.) following manufacturers' instructions. The reactivities of biotin-labeled mAbs were validated by flowcytometry. All biotin-conjugated mAbs showed homogeneous, HLA-allele-specific staining on CD3 positive cells.

### 1.1.4 Flowcytometric Analyses

HLA class I cell surface expression was determined by labeling with biotinylated human HLA-specific mAbs counterstained with streptavidin-phycoerythrin (Becton Dickinson) in appropriate dilutions. Gates were set on vital lymphocytes according to their typical forward- and side-scattering characteristics. All flowcytometric analyses were performed on a FACSCalibur with Cellquest software (Becton Dickinson). Results are displayed as mean fluorescence intensity (MFI).

### 1.1.5 Cytotoxicity Assays

Cytotoxicity was evaluated by incubating 2500 ⁵¹Cr labeled target cells with serial dilutions of CTLs for 4 hours. Supernatants were harvested for gamma counting. % specific lysis=(experimental release-spontaneous release)/(maximal release-spontaneous release)×100%. Results are expressed as the mean of duplicate samples and shown for an effector:target ratio of 10:1 unless stated otherwise.

### 1.1.6 Statistics

Statistical analyses were performed using unpaired t-tests for data derived from single experiments and paired t-tests for data pooled from multiple experiments. P values <0.05 were considered to be significant. Data pooled from multiple experiments were standardized as follows. Fluorescence (in fluorescence units): (mean fluorescence−mean fluorescence of isotype control)/(mean fluorescence of mock control−mean fluorescence of isotype control); lysis: mean % lysis/mean % lysis mock. Error bars represent standard errors of the mean.

### 1.2. Results

### 1.2.1 Effects of US6, ICP47 and UL49.5 on HLA Class I Cell Surface Levels

EBV-LCLs derived from HLA-A2pos, HLA-B60pos donor Modo (Table I), were retrovirally transduced with US6, ICP47 or UL49.5, or with an empty control vector to evaluate the effects of the three TAP-inhibitors on HLA class I expression and antigen-presentation. Cell surface levels of HLA-A2 and HLA-B60 were analyzed using HLA allele-specific mAbs (data not shown). The HLA-A2 expression of EBV-LCLs transduced with US6, ICP47 or UL49.5 decreased with 63%, 57% and 73%; the HLA-B60 expression with 80%, 82% and 99%, compared to the empty vector-transduced EBV-LCL (P<0.05). These low HLA class I cell surface levels remained consistent upon continuous in-vitro culture (data not shown). No difference in HLA-A2 or HLA-B60 expression could be observed between untransduced and empty vector-transduced EBV-LCLs.

1.2.2 Effects of US6, ICP47 and UL49.5 on mHag-Specific Target Cell Recognition

To determine whether the downregulation of HLA class I cell surface expression resulted in a decrement of functional recognition by mHag-specific CTLs, the transduced Modo EBV-LCLs were used as target cells in cytotoxicity assays. Four different CTL clones with previously established specificity for the mHags (HLA-) A2/HA-1, A2/HA-2, A2/HY, or B60/HY, were used as effector cells (data not shown). The Modo EBV-LCLs naturally express each of these mHags (Table 1). All CTL clones exhibited a significantly diminished recognition of TAP-inhibitor-transduced EBV-LCLs as compared to empty vector-transduced EBV-LCL (P<0.05). Inhibition of target cell lysis ranged from 70% to 87% for US6, 77% to 89% for ICP47, and 85% to 99% for UL49.5 for the various CTL clones. Increasing the E:T ratio did not restore target cell recognition (data not shown), indicating consistent blocking of endogenous mHag-peptide translocation and HLA-loading by TAP-inhibitors. No difference could be detected between untransduced and control-transduced EBV-LCLs for any of the CTL clones tested.

TAP-inhibiting effects by US6, ICP47, and UL49.5 were statistically analyzed by pooling the data on HLA-A2 and HLA-B60 expression as well as the data on mHag-recognition by the various CTL clones from the experiments described above. This analysis showed significant differences for decrement of HLA class I expression between US6 and UL49.5 (P=0.0264), and ICP47 and UL49.5 (P=0.0006), but not between US6 and ICP47 (P=0.6474). Similarly, decreases in mHag-specific lysis differed significantly between US6 and UL49.5 (P=0.0005), and ICP47 and UL49.5 (P=0.0346), but not between US6 and ICP47 (P=0.1355). These results seem to indicate that UL49.5 is consistently more effective in downregulating endogenous mHag-presentation.

### 1.2.3 Effects of Exogenous Peptide-Addition on Recognition of Tap-Inhibited Target Cells

TAP inhibitory proteins affect HLA class I expression because the absence of endogenous peptide renders HLA class I molecules expressed at the cell surface unstable. Yet, HLA class I cell surface expression is not completely abrogated. Exogenously added peptide can bind to these HLA class I molecules. To investigate whether sufficient HLA class I molecules remain for functional mHag presentation, we loaded TAP-inhibitor transduced EBV-LCLs with mHag peptides. Hereto, HLA-A2pos HA-1pos Modo EBV-LCLs transduced with US6, ICP47, UL49.5, or an empty vector, were pulsed with various concentrations of HA-1 peptide. An EBV-LCL derived from HLA-A2pos HA-1neg donor H6 (Table I) was included as a control (data not shown). Addition of HA-1 peptide to TAP-inhibited EBV-LCLs restored recognition by A2/HA-1-specific CTLs in a dose-dependent manner to the level observed for the control target cell H6. Addition of non-specific peptide had no effect (data not shown). Thus, even low numbers of HLA molecules appear to be sufficient for functional mHag-specific recognition. Thus, upon functional inhibition of TAP, the target cell can still be pulsed exogenously with any HLA-binding peptide of interest; one of the original aims of our study.

2.4 Effects of TAP-Inhibition on alloHLA-A2-Specific Target Cell Recognition

As mentioned above, TAP-inhibition does not abrogate cell surface HLA class I expression completely. Thus, TAP-inhibited EBV-LCLs may still present peptides on the cell surface that can be recognized by alloHLA-specific CTLs. To test the latter hypothesis, we compared alloHLA-recognition of empty vector-transduced EBV-LCL and TAP-inhibited EBV-LCLs. EBV-LCLs derived from HLA-A2pos donor Modo and transduced with US6, ICP47, UL49.5 or an empty vector were used as targets in a cytotoxicity assay. As effector cells, we used two alloHLA-A2 specific CTL clones (designated clone #1 and clone #2). Clone #1 was shown to be TAP-dependent in earlier experiments (data not shown), whereas clone #2 is known to be TAP-independent (31). The HLA-A2pos TAP-deficient cell line T2 was included as a control. Two E:T ratios are shown for the transduced Modo EBV-LCLs i.e. 10:1 and 1:1 (data not shown). TAP-dependent CTL clone #1 exhibited a significantly diminished recognition of ICP47- and UL49.5-transduced EBV-LCLs as compared to empty vector-transduced EBV-LCL for both E:T ratios (P<0.05). No significant inhibition of lysis was observed for US6 in this experiment (P=0.05). Of ICP47 and UL49.5, the latter was again the more potent inhibitor (P<0.05 E:T1:1, P=0.33 E:T 10:1). AlloHLA-A2 recognition of UL49.5-transduced Modo EBV-LCL was reduced by 90% for E:T ratio 1:1 but only by 47% for E:T ratio 10:1. In comparison, at E:T ratio 10:1 mHag-specific recognition of UL49.5-transduced Modo-EBV-LCLs was abrogated almost completely (data not shown). Apparently, UL49.5 does not inhibit the presentation of peptides recognized by TAP-dependent alloHLA-A2-specific CTLs completely. AlloHLA-A2 recognition by TAP-independent CTL clone #2 was not affected by any of the TAP-inhibitors.

2.5 Effects of UL49.5 on alloHLA-A1-Specific Target Cell Recognition

Inhibition of TAP has been reported to have a stronger effect on HLA-A1-expression than on HLA-A2-expression (32, 33). Therefore, we also evaluated alloHLA-A1-specific recognition of UL49.5-transduced EBV-LCLs. To that end, we retrovirally transduced EBV-LCLs derived from HLA-A1pos donor Hodo with UL49.5 or with an empty control vector. UL49.5-transduced Hodo EBV-LCL showed significantly decreased cell surface HLA-A1 expression (P<0.05) and decreased susceptibility to lysis by an HLA-A1-restricted HY-specific CTL clone (data not shown). Ul49.5- and control-transduced Hodo EBV-LCLs were then used as targets for an alloHLA-A1-specific CTL clone (designated clone #3) as effector cell. E:T ratios 10:1 and 1:1 are shown for the transduced Hodo EBV-LCLs (data not shown). AlloHLA-A1-specific recognition was significantly decreased for UL49.5-expressing Hodo EBV-LCLs as compared to control-transduced EBV-LCLs (P<0.05). However, downregulation of alloHLA-A1-specific lysis was not complete, similar to that of alloHLA-A2-specific lysis. Taken together, these findings indicate that retroviral transduction of APCs with UL49.5 diminishes but not abrogates major alloHLA-recognition in a TAP-dependent fashion.

### 2. Example 2

### UL49.5 Regulates the Presentation of Murine CTL Epitopes by Qa-1b

### 2.1 Materials and Methods

2.1.1 Cell lines The tumor cell lines used in this study have been generated by chemical carcinogens in different mouse strains. Coloncarcinoma C26 and CC36 were derived from the BALB/c stain and MC38 was derived from the C57BL/6 strain (34). Introduction of the UL49.5 gene from bovine herpesvirus 1 (BHV1) was established by retroviral gene transduction with the LZRS vector containing an IRES GFP, as described before (21). Cells with the highest GFP expression were positively sorted by FACS. Fibrosarcoma MCA was generated in the TAP1−/− mouse on C57BL/6 background (24). TAP1 restoration in this cell line was performed with a retroviral construct encoding the mouse TAP1 gene, as described (24). CTL clone E/88 recognizes the H-2Ld-binding peptide SPSYVYHQF comprised in an endogenous retroviral gp70 gene product and was generously provided by Dr. M. Colombo (35). These CTL were weekly restimulated with irradiated C26 tumor cells together with 10 Cetus Units recombinant human IL-2 (Cetus, Amsterdam, the Netherlands). CTL clone D12i recognizes the H-2 Db-derived leader peptide AMAPRTLLL in the context of Qa-1b and was generously provided by Dr. C. Brooks (36). These CTL were generated in B6.Tla mice that harbor the Qa-1a allotype and were weekly restimulated with irradiated B6 spleen cells and IL-2. Generation of TEIPP-specific Qa-1b-restricted CTL have been described before (24). These CTL were weekly restimulated with irradiated B7.1 expressing EC7.1.Qa-1b cells, irradiated spleen cells and IL-2. All cell lines were cultured in Iscove's modified Dulbecco's medium (Biowhittaker Europe, Verviers, Belgium), supplemented with 8% heat-inactivated foetal calf serum (Gibco BRL, Breda, the Netherlands), 2 mM L-glutamine (ICN Biomedicals Inc., Costa Mesa, Calif.), 100 IU/ml penicillin (Yamanouchi Pharma, Leiderdorp, the Netherlands), and 30 μM 2-mercapto-ethanol (Merck, Darmstadt, Germany) at 37° C. in humidified air with 5% CO2.

### 2.1.2 CTL Activation Assay and Flowcytometry

Graded amounts of target cells were incubated with 5×103 CTL in round-bottom 96-well plates. After 18 h of co-culture, supernatants were measured for IFN-γ content using sandwich ELISA, as described before (37). Surface expression of MHC class I molecules was determined using mouse anti-Qa-1b mAb (clone 6A8, Pharmingen) and mouse anti-Ld mAb (clone 28-14-8, Pharmingen) followed with APC-labelled goat-anti-mouse Ig and analyzed on a FACS Callibur machine (Becton Dickinson).

### 2.1.3 Peptide Transport Assays

### 2.1.3.1 Human Cells

Cells were permeabilized by using 2.5 units/ml Streptolysin O (Murex Diagnostics, Dartford, U.K.) at 37° C. for 15 min (40). Permeabilized cells (2×10⁶ cells per sample) were incubated with 10 μl (≈100 g) of ¹²⁵I-labeled peptide (41) or 10 μl (≈200 pmol/μl) of fluorescein-conjugated synthetic peptide CVNKTERAY in the presence or absence of ATP (10 mM final concentration) at 37° C. for 10 min. Peptide translocation was terminated by adding 1 ml of ice-cold lysis buffer (1% Triton X-100/500 mM NaCl/2 mM MgCl₂/50 mM Tris.HCl, pH 8). After centrifugation at 12,000×g, supernatants were collected and incubated with 100 μl of ConA-Sepharose (Amersham Pharmacia) at 4° C. for 1 h to isolate the glycosylated peptides. The beads were washed and the peptides were eluted in the presence of elution buffer (500 mM mannopyranoside/10 mM EDTA/50 mM Tris.HCl, pH 8) by rigorous shaking at 25° C. for 1 h. Radioactivity was measured by gamma counting. Fluorescence intensity was measured with a fluorescence plate reader (CytoFluor, PerSeptive Biosystems, Framingham, Mass.) with excitation and emission wavelengths of 485 and 530 nm, respectively. Peptide transport is expressed as percentage of translocation, relative to the translocation observed in control cells (set at 100%).

### 2.1.3.2 Murine Cells

Mouse coloncarcinoma cells (2.5×10⁶ cells per assay) were semipermeabilized with saponin (0.05% (w/v)) in 50 μl of AP-buffer (PBS with 5 mM MgCl₂) for 1 min at room temperature. Cells were washed twice with AP-buffer. Peptide transport assays were performed with 0.46 μM of fluorescein-labeled peptide (RRYQNSTCfL, N-core glycosylation site underlined) in AP-buffer (total volume of 100 μl per assay) in the presence of 10 mM of ATP for 3 min at 32° C. Apyrase (1 U, Sigma) was added to deplete ATP in the control samples. The transport reaction was terminated by addition of 1 ml stop-buffer (PBS with 10 mM EDTA). Cells were then collected by centrifugation and lysed in buffer (50 mM Tris/HCl, 150 mM NaCl, 5 mM KCl, 1 mM CaCl2, 1 mM MnCl2, 1% NP40; pH 7.5) for 20 min on ice. The N-core glycosylated peptides were recovered with concanavalin A (ConA)-sepharose beads (Sigma) overnight at 4° C. After washing with lysis buffer, peptides were eluted from the sepharose beads with 200 mM of methyl-α-D-mannopyranoside and quantified with a fluorescence plate reader (λex/em=485/520 nm; Polarstar Galaxy, BMG).

### 2.1.4 Immunoblotting

Cells (5×106 cells) were lysed in NP40 lysis buffer (1% NP40 in 50 mM Tris-HCl, 50 mM NaCl, 5 mM MgCl2, pH 7.4) and mixed with SDS sample buffer without boiling. Proteins were separated by SDS-PAGE and transferred to nitrocellulose membranes (Schleicher and Schuell). Membranes were saturated with skimmed milk powder (2% w/v) and then probed with a β-actin-specific antibody (Sigma), a mouse TAP2-specific serum (TAP2.688, a kind gift of Dr. F. Momburg), or a mouse TAP1-specific mAb (clone SC-11465, Santa Cruz). After washing with TBS (0.1% Tween in PBS), bound primary antibodies were detected using peroxidase conjugated antibodies: goat anti-mouse Ig (SouthernBiotech), a goat anti-rabbit Ig (SouthernBiotech) and rabbit anti-goat Ig (DAKO), respectively. After washing with TBS, peroxidase activity was visualized by chemiluminescence imaging (Lumi-Imager F1TM, Roche).

### 2.2 Results

### 2.2.1 UL49.5 Protein Impairs TAP Function in Mouse Cells

We previously reported that the BHV1-derived molecule UL49.5 is accountable for the inactivation of TAP through inducing conformational changes and active breakdown of this peptide transporter (21). These studies were performed in human cell lines. Here, we introduced UL49.5 in cells of mouse origin to examine whether this protein also inactivates mouse TAP. Expression of UL49.5 resulted in marked reduction of MHC class I surface display, irrespective of the MHC haplotype or the tissue origin of the cells (H-2b, H-2d and H-2k) (data not shown). These findings indicated that UL49.5, in addition to bovine and human TAP, also inhibits peptide transport by mouse TAP. Inhibition of TAP function was more directly assessed in a peptide transport assay using a labeled prototypic peptide. Indeed, a strong reduction in peptide transport activity was observed in cells that expressed the viral UL49.5 gene (data not shown). Compared to control transfected cells, TAP activity was 3 to 5 fold decreased. Finally, the influence of the TAP inhibition on the processing and presentation of a characterized peptide-epitope was evaluated. The surface presentation of the Ld-binding peptide SPSYVYHQF was determined using a peptide-specific CTL clone (35). This peptide is derived from an endogenous tumor antigen that is expressed in colon carcinomas (35). IFNγ release by the CTL was measured upon co-incubation with the Ld-expressing coloncarcinoma cell lines C26 and CC36 expressing UL49.5 or a control construct. Four to six times more UL49.5-positive target cells were needed to reach similar IFNγ levels, showing that UL49.5-mediated inhibition of TAP has functional consequences for antigen presentation to CTL (data not shown). Collectively, these data show that the BHV1 UL49.5 protein inhibits peptide transport by TAP in mouse cells.

### 2.2.2 Inactivation of TAP is Exerted Through Degradation

Our previous studies in human cells revealed that UL49.5 inactivates TAP via two mechanisms. The binding of UL49.5 to TAP results in a translocation-incompetent state of the transporter complex. Ultimately, UL49.5 mediates the degradation of both TAP subunits via the proteasome (21). UL49.5 breakdown coincides in this process. This mechanism is clearly different from that of other viral proteins that disturb the peptide transport process. To determine if mouse TAP1 and TAP2 are similarly destabilized by UL49.5, we examined their steady state levels in immunoblots (data not shown). Reduced levels of both TAP1 and TAP2 were observed in the cells that expressed UL49.5 compared to their counterparts (data not shown), indicating that UL49.5 destabilizes both subunits. A comparable decrease was observed in all three coloncarcinoma cell lines analyzed, explaining the observed general reduction in MHC class I display at the cell surface (data not shown). These results are in line with our previous findings in human cells and argue that UL49.5 interacts with human and mouse TAP1/TAP2 heterodimers at a region that is structural homologous.

### 2.2.3 Peptide Presentation by the Non-Classical MHC Class I Molecule Qa-1B

To date, UL49.5 is the first protein that can efficiently inhibit TAP function in multiple species, including mouse. We anticipate that this feature of UL49.5 makes it a very suitable research tool for application in diverse mouse systems of antigen processing and presentation. We examined the influence of UL49.5 on the peptide repertoire that is presented by the non-classical class I molecule Qa-1b. We analyzed the Qa-1b-mediated presentation of a TAP-dependent leader peptide (AMAPRTLLL) that is derived from the classical MHC class I molecule H-2 Db (36) and the recently determined TEIPP peptides (24) using Qa-1b-restricted CTL clones. TEIPP represents a novel set of CTL epitopes that are selectively presented by cells with antigen processing defects, such as TAP-deficient tumors (24).

Fibrosarcoma cells (MCA) from a TAP1−/− mouse failed to trigger AMAPRTLLL-specific CTL (FIG. 3, left panel). Gene transfer of mouse TAP1 restored the presentation of this peptide, while IFNγ treatment in addition to TAP1 expression further augmented the CTL reactivity (data not shown). In contrast, Qa-1b-restricted CTL with TEIPP specificity were activated by the TAP-deficient variant and TAP restoration decreased the CTL response (data not shown). Promotion of antigen processing by pre-treatment with IFNγ resulted in even lower CTL responses. This pattern of CTL recognition is in line with our previous results on H-2 Db- and H-2 Kb-restricted TEIPP CTL, as target cells with impaired antigen processing efficiently stimulate TEIPP CTL (24). Thus, these data on genetic TAP-deficient cells revealed opposing requirements of these two Qa-1b-presented peptides for the intracellular processing machinery, in that the leader peptide AMAPRTLLL depends on TAP function and TEIPP peptides benefit from TAP deficiency.

2.2.4 The presentation of Qa-1b-Binding Tap-Independent Epitopes is Promoted by UL49.5

Next, we analyzed Qa-1b-mediated antigen presentation by the three TAP-positive coloncarcinoma cell lines (C26, CC36 and MC38, data not shown). All three cell lines display the same Qa-1 allele (Qa-1b), although they are derived from different mouse strains (BALB/c and C57BL/6) (34). Qa-1 genes display very limited polymorphism, in fact only two different alleles have been described thus far (38). This allowed us to use the same Qa-1b-restricted CTL clones for the analysis of the Qa-1 peptide repertoire. In accordance with the TAP1-deficient fibrosarcoma experiments (data not shown), the recognition by the AMAPRTLLL peptide-specific CTL was clearly decreased upon expression of UL49.5 (data not shown). The TEIPP-specific CTL did not respond against the parental C26, CC36 or MC38 cells (data not shown), suggesting that the TAP function in these cells precluded the presentation of TEIPP epitopes. Interestingly, UL49.5 expression induced the emergence of TEIPP at the cell surface of the coloncarcinomas and strongly promoted activation of the TEIPP-specific T-cells (data not shown). These findings imply that, in the absence of functional TAP, peptides other than MHC class I-derived leader peptides substitute the Qa-1b-binding peptide pool. Overall Qa-1b surface levels were not affected by the UL49.5 protein (data not shown), underlining the notion that UL49.5 selectively attacks the antigen processing machinery and does not limit the availability of Qa-1 heavy chains.

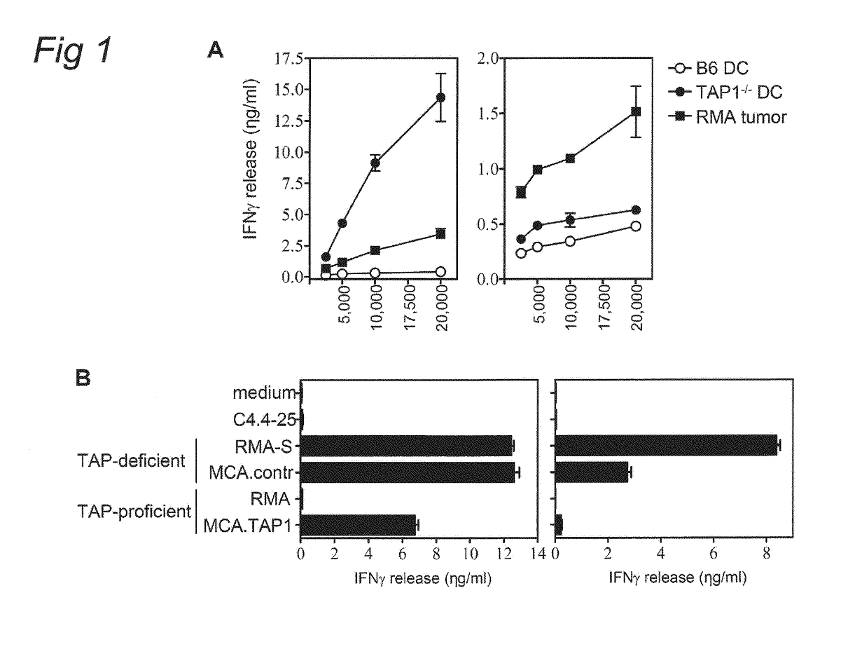

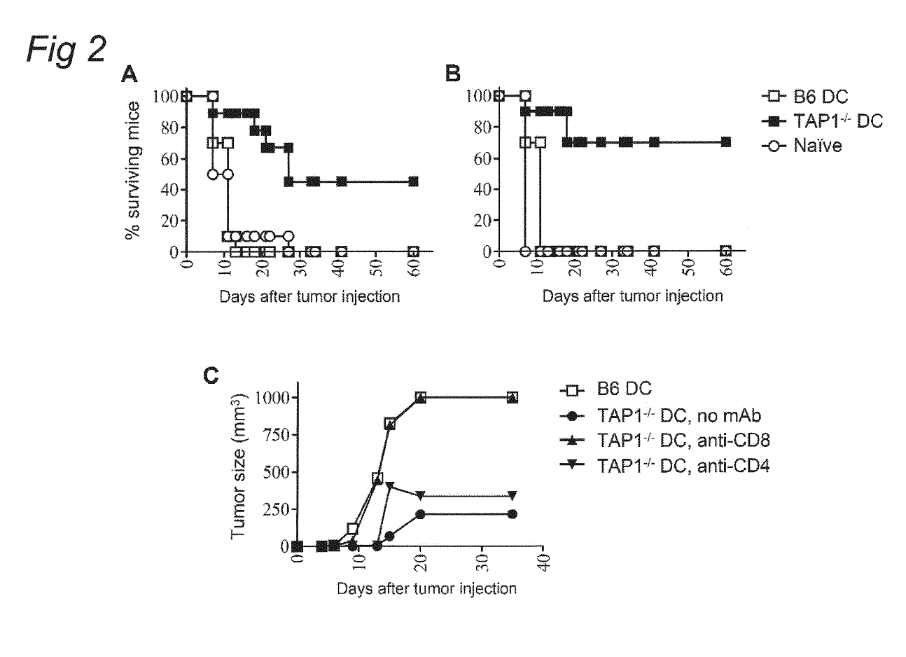

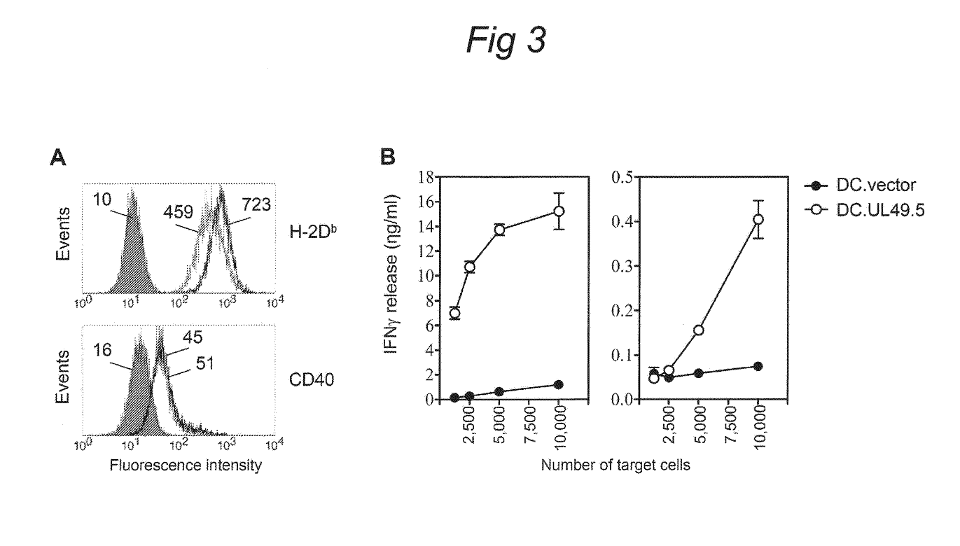

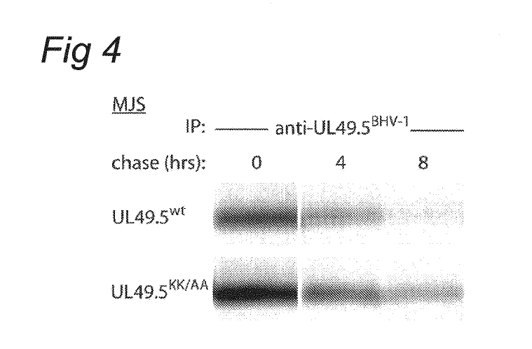

IFNγ strongly enhances the class I antigen processing and presentation machinery. We assessed if pre-treatment of IFNγ would reduce the UL49.5-mediated display of TEIPP antigens by Qa-1b. This is of interest since the UL49.5 protein seems to block peptide transport and subsequent presentation only partially (FIG. 1B-C, and compare FIG. 3 with FIG. 4A). Treatment of CC36 cells with IFNγ resulted in improved presentation of the TAP-dependent AMAPRTLLL peptide (FIG. 5, left panel). Similar CTL recognition patterns were observed against targets that had not been pre-treated with IFNγ (FIG. 5, left panel). The impact of UL49.5 was comparable with that of non-treated target cells. Strikingly, the reactivity of TEIPP-specific CTL was not affected by IFNγ treatment of the target cells, indicating that UL49.5 function was sufficient to counteract the augmented antigen processing. Together, our data show that the varicellovirus derived protein UL49.5 is an efficient TAP inhibitor in mouse cells and may be exploited as a versatile tool for the induced presentation of TEIPP antigen.

### 3. Example 3

### Dendritic Cells Deficient for the Peptide Transporter Tap Arouse Protective CTL Immunity Against Tumor Immune Escape Variants

### 3.1 Material and Methods

### 3.1.1 Cell Lines and Mice

Tumor cell lines used in this study, RMA-S lymphoma and MCA fibrosarcoma were described before (24). D1 cells are growth factor-dependent immature dendritic cells and were kindly provided by Dr. F. Ossendorp (39). Mouse TAP1 gene and the Bovine Herpes Virus-1 derived gene UL49.5, which was kindly provided by Dr. E. Wiertz, were cloned into retroviral plasmid vector LZRS and gene transduction was performed as previously described (in Example 2 and 2 1). In this study several TEIPP-specific CTL clones are used: c1G, c1B5 and mi3. All display similar specificity for TAP-deficient target cells. Tumor-specific CTL clone c117 recognizes the peptide NKGENAQAI as presented by RMA cells (37). CTL were weekly restimulated with irradiated tumor cells (RMA-S.B7 and RMA, respectively) together with 10 Cetus Units recombinant human IL-2 (Cetus, Amsterdam, the Netherlands) and irradiated naïve splenocytes. All cell lines were cultured in Iscove's modified Dulbecco's medium (Biowhittaker Europe, Verviers, Belgium), supplemented with 8% heat-inactivated foetal calf serum (Gibco BRL, Breda, the Netherlands), 2 mM L-glutamine (ICN Biomedicals Inc., Costa Mesa, Calif.), 100 IU/ml penicillin (Yamanouchi Pharma, Leiderdorp, the Netherlands), and 30 μM 2-mercapto-ethanol (Merck, Darmstadt, Germany) at 37° C. in humidified air with 5% CO₂. C57BL/6 (B6) mice and TAP1−/− mice on B6 background were bred in the in-house facility under SPF conditions of the Microbiology and Tumorbiology Center of the Karolinska Institutet, Stockholm. All experiments in this study received approval from the local ethical committee of the Karolinska Institutet in Stockholm.

### 3.1.2 CTL Activation Assays and Flowcytometry

Assays to measure CTL activity, the release of IFNγ by ELISA and cytotoxic capacity by 51Cr-labelled targets, were described before (21). Expression levels of cell surface molecules were determined with flowcytometry using a FACSCalibur™ (Becton Dickinson). The following antibodies were used: anti-Db (clone 28-14-8, BD), anti-CD40 (clone 3/23, BD), anti-CD80 (Clone 16-10A1, BD), anti-CD86 (Clone 24F, BD).

### 3.1.3 Preparation of Dendritic Cells

Bone marrow harvested from femurs of B6 mice. Cultured with GM-CSF for 10 days in the presence of normal mouse serum. Purity of the cultures and successful maturation by IFNγ were analyzed by flowcytometry. All cultures contained at least 90% CD11c-positive cells and expression of maturation markers CD40, CD80, CD86 and MHC class II were clearly upregulated compared to cells that were not treated with IFNγ (data not shown).

### 3.1.4 Tumor Protection Experiments

DC were prepared as described. Spleens harvested and after two restimulated with RMA-S.B7 tested against target cells. For tumor protection experiments, After two i.v. administrations of the dendritic cells, tumor suspensions were injected s.c. twice in 14 days, tumors that had been passage ip in mice. Tumors were measured twice a week and mice were euthanized when tumors reached a volume of 1000 mm3. Prevention of foetal calf serum component in tumors and DC was crucial to exclude FCS derived foreign antigens. Repeated injections with anti-NK1.1 (clone PK136) in order to deplete NK cells to prevent NK-mediated kill of tumor cells.

### 3.2 Results

### 3.2.1 TAP-Deficient Dendritic Cells Induced TEIPP-Specific CTL

Previously, we demonstrated the existence of a, thus far unknown, population of anti-tumor CTL that are capable to eradicate tumor cells with antigen processing deficiencies (24). Their cognate peptide-epitopes (which we refer to as TEIPP, standing for ‘T cell epitopes associated with impaired peptide processing’) are derived from widely distributed ‘self’ proteins, but are not presented by MHC class I on cells with normal antigen processing function (24). In view of the crucial role of dendritic cells in the initiation of T-cell responses and their application in anti-cancer vaccines, we analyzed the display of TEIPP peptides by dendritic cells with impaired TAP function. Bone marrow-derived dendritic cells from TAP1−/− mice, but not from wild type mice, were efficiently recognized by previously established TEIPP-specific CTL (FIG. 1A). Control CTL directed against RMA lymphoma cells were not stimulated by the dendritic cell populations (FIG. 1A). These findings prompted us to test the in vivo capacity of these autologous dendritic cells to induce TEIPP-specific CTL responses. Mature dendritic cells from TAP1−/− mice were injected into syngeneic mice and the cytolytic activity in the spleens of recipient mice was tested against a panel of tumor cells: TAP-deficient RMA-S cells, TAP-positive RMA counterparts and β2m-negative C4.4-25 control cells (Table 2). All cultures displayed preferential kill of RMA-S cells, while reactivity against C4.4-25 was generally low, indicating that the immunization strategy indeed resulted in the induction of TEIPP CTL responses. NK cells, which also exhibit preferential kill of MHC class Ilow RMA-S targets, were depleted in vivo to exclude potential confounding reactivity. Together, these results indicate that dendritic cells are able to generate TEIPP-specific CTL responses in vivo.

TABLE 2
Immunization with TAP-deficient DC induces TEIPP-specific
CTL responses

| | | CTL culture | | | | |
|---|---|---|---|---|---|---|
| Target cell^{a} | E:T ratio^{b} | #1 | #2 | #3 | #4 | #5 |
| RMA-S | 40:1 | 62 ± 3°^{c} | 62 ± 5 | 54 ± 3 | 32 ± 4 | 39 ± 4 |
| | 20:1 | 54 ± 5 | 48 ± 2 | 43 ± 2 | 28 ± 3 | 31 ± 1 |
| | 10:1 | 42 ± 2 | 44 ± 4 | 32 ± 2 | 20 ± 2 | 21 ± 1 |
| | 5:1 | 26 ± 1 | 27 ± 2 | 21 ± 1 | 10 ± 0 | 16 ± 1 |
| RMA | 40:1 | 26 ± 5 | 53 ± 0 | 29 ± 3 | 26 ± 4 | 26 ± 1 |
| | 20:1 | 25 ± 2 | 40 ± 2 | 23 ± 2 | 20 ± 2 | 21 ± 0 |
| | 10:1 | 19 ± 0 | 38 ± 3 | 17 ± 2 | 15 ± 1 | 19 ± 1 |
| | 5:1 | 13 ± 2 | 30 ± 1 | 13 ± 1 | 11 ± 1 | 14 ± 1 |
| C4.4-25 | 40:1 | 34 ± 4 | 24 ± 0 | 19 ± 2 | 10 ± 3 | 10 ± 0 |
| | 20:1 | 22 ± 3 | 29 ± 2 | 14 ± 1 | 8 ± 0 | 13 ± 2 |
| | 10:1 | 18 ± 2 | 21 ± 4 | 9 ± 2 | 2 ± 1 | 6 ± 2 |
| | 5:1 | 10 ± 2 | 11 ± 2 | 7 ± 2 | 3 ± 1 | 1 ± 2 |

^{a}RMA-S is a TAP2 defiicient lymphoma, RMA is the TAP-proficient counterpart, C4.4-25 is a β2m-deficient lymphoma (see Material and Methods for details)
^{b}E:T ratio, effector to target cell ratio
^{c}Shown are means specific lysis of triplicate wells ± standard deviation

3.2.2 Immunization with TAP-Deficient Dendritic Cells Protects Mice Against TAP-Negative Tumors

On basis of these data, we tested whether vaccination with TAP-deficient dendritic cells was strong enough to protect mice for outgrowth of tumor immune escape variants. Two different tumors types were chosen for this examination, the TAP2-deficient RMA-S lymphoma and the TAP1-deficient MCA fibrosarcoma. Both tumors were strongly recognized by previously established CTL clones with TEIPP-specificity, whereas the reactivity against TAP-expressing counterpart tumors was less efficient (FIG. 1B). The observation that tumors of completely different tissue origins are recognized by the same TEIPP CTL clones, illustrates the fact that their cognate peptide-epitopes are derived from widely distributed ‘self’ proteins, like the ceramide regulator Trh4 (24).

Normal wild type mice were injected with syngeneic bone marrow-derived dendritic cells from TAP1−/− or wild type mice and challenged with a lethal dose of TAP-deficient RMA-S (FIG. 2A) or MCA (FIG. 2B) tumors. All mice that received salt solution or wild type dendritic cells developed tumors and had to be sacrificed within three weeks due to progressively growing lesions (FIG. 2A). In contrast, mice that received TAP1−/− dendritic cells showed delayed tumor growth and 40 to 70 percent of the mice (for RMA-S and MCA, respectively) were completely protected against tumor outgrowth. Of note, NK cells were depleted during the complete course of these experiments in order to exclude the possibility that protective capacity relied in the NK compartment. To further substantiate this conclusion, we performed in vivo depletion studies using anti-CD4 and anti-CD8 antibodies (FIG. 2C). Clearly, CD8+ T-cells were accountable for the prevention of RMA-S tumor outgrowth. In conclusion, our data indicate that TAP-deficient dendritic cells can mediate protection against processing deficient tumors through the in vivo activation of TEIPP CTL responses.

### 3.2.3 Viral Inhibitors of Tap Function Induce the Presentation of TEIPP

Dendritic cells with genetic loss of TAP1 have thus far been employed in our studies. Application of this concept in the clinic would, however, involve autologous dendritic cells that are rendered TAP deficient. In order to examine the feasibility of such an approach, we made use of an immune evasion protein from Bovine Herpes Virus-1, that we recently demonstrated to inhibit TAP function in human as well as mouse cells (Example 2 and 21). This viral UL49.5 gene was introduced into dendritic cell line D1 via a retroviral expression system. Expression of UL49.5 resulted in a 40% to 50% reduction of surface MHC class I display (FIG. 3A, upper panel), indicating that the inhibitor strongly, but not completely, impaired TAP-mediated transport of peptides. No alterations were observed in the surface display of other molecules, like CD40 (FIG. 3A, bottom panel). Importantly, TEIPP-specific CTL clones responded selectively against UL49.5-expressing dendritic cells (FIG. 3B), indicating that TEIPP peptides are indeed induced in dendritic cells upon blocking TAP function. The UL49.5 protein therefore constitutes a formidable tool for the arousal of TEIPP-directed CTL responses in the immune control of tumor escape variants.

### Example 4

### Substitution of Lysine Residues in the Cytoplasmic Tail of BHV1-UL49.5 Increases the Stability of the Viral Inhibitor

MJS cells were transduced with retroviruses to express wild type UL49.5 or a recombinant form in which the two lysine residues in the cytoplasmic tail (positions 68 and 69 in SEQ ID NO.1) have been substituted for alanines. To evaluate the stability of these proteins, the cells were pulse-labeled with [35]S-methionine/cysteine and chased for 4 and 8 hrs. UL49.5 wt and UL49.5 KK/AA were immunoprecipitated from cell lysates, separated on SDS-PAGE and displayed using phosphoimaging technology. This pulse-chase experiment indicates that the stability of UL49.5 can be increased in cells by replacing its two cytoplasmic tail lysine residues by alanines. However, inactivation of TAP through degradation is not affected by these modifications in the cytoplasmic tail of UL49.5 (data not shown).

TABLE 3
Codon Frequency in Highly Expressed Human Genes

| | | % occurance |
|---|---|---|
| Ala | | |
| GC | C | 53 |
| | T | 17 |
| | A | 13 |
| | G | 17 |
| Arg | | |
| CG | C | 37 |
| | T | 7 |
| | A | 6 |
| | G | 21 |
| AG | A | 10 |
| | G | 18 |
| Asn | | |
| AA | C | 78 |
| | T | 25 |
| Leu | | |
| CT | C | 26 |
| | T | 5 |
| | A | 3 |
| | G | 58 |
| TT | A | 2 |
| | G | 6 |
| Lys | | |
| AA | A | 18 |
| | G | 82 |
| Pro | | |
| CC | C | 48 |
| | T | 19 |
| | A | 16 |
| | G | 17 |
| Thr | | |
| AC | C | 57 |
| | T | 14 |
| | A | 14 |
| | G | 15 |
| Tyr | | |
| TA | C | 74 |
| | T | 26 |
| Cys | | |
| TG | C | 68 |
| | T | 32 |
| Gln | | |
| CA | A | 12 |
| | G | 88 |
| Glu | | |
| GA | A | 25 |
| | G | 75 |
| Gly | | |
| GG | C | 50 |
| | T | 12 |
| | A | 14 |
| | G | 24 |
| His | | |
| CA | C | 79 |
| | T | 21 |
| Ile | | |
| AT | C | 77 |
| | T | 18 |
| | A | 5 |
| Ser | | |
| TC | C | 28 |
| | T | 13 |
| | A | 5 |
| | G | 9 |
| AG | C | 34 |
| | T | 10 |
| Phe | | |
| TT | C | 80 |
| | T | 20 |
| Val | | |
| GT | C | 25 |
| | T | 7 |
| | A | 5 |
| | G | 64 |

### REFERENCES

1. Harty, J. T., A. R. Tvinnereim, and D. W. White. 2000. CD8+ T cell effector mechanisms in resistance to infection. Annu Rev Immunol 18:275.
2. Melief, C. J. M., R. E. Toes, J. P. Medema, S. H. Van der Burg, F. Ossendorp, and R. Offring a. 2000. Strategies for immunotherapy of cancer. Adv. Immunol. 75:235.
3. Offring a, R. 2005. Tumour immunology exploitation of the weapon of immune destruction for cancer therapy: taking aim before firing. Curr. Opin. Immunol. 17:159.
4. Dudley, M. E., J. R. Wunderlich, P. F. Robbins, J. C. Yang, P. Hwu, D. J. Schwartzentruber, S. L. Topalian, R. Sherry, N. P. Restifo, A. M. Hubicki, M. R. Robinson, M. Raffeld, P. Duray, C. A. Seipp, L. Rogers-Freezer, K. E. Morton, S. A. Mavroukakis, D. E. White, and S. A. Rosenberg. 2002. Cancer regression and autoimmunity in patients after clonal repopulation with antitumor lymphocytes. Science 298:850.
5. Offring a, R., S. H. Van der Burg, F. Ossendorp, R. E. Toes, and C. J. Melief. 2000. Design and evaluation of antigen-specific vaccination strategies against cancer. Curr. Opin. Immunol. 12:576.
6. Rosenberg, S. A., J. C. Yang, and N. P. Restifo. 2004. Cancer immunotherapy: moving beyond current vaccines. Nat. Med. 10:909.
7. Gottschalk, S., H. E. Heslop, and C. M. Rooney. 2005. Adoptive immunotherapy for EBV-associated malignancies. Leuk Lymphoma 46:1.
8. Vossen, M. T., E. M. Westerhout, C. Soderberg-Naucler, and E. J. Wiertz. 2002. Viral immune evasion: a masterpiece of evolution. Immunogenetics 54:527.
9. Wiertz, E. J., S. Mukherjee, and H. J. Ploegh. 1997. Viruses use stealth technology to escape from the host immune system. Mol. Med. Today 3:116.
10. Gamido, F., and I. Algarra. 2001. MHC antigens and tumor escape from immune surveillance. Adv Cancer Res 83:117.
11. Hicklin, D. J., F. M. Marincola, and S. Ferrone. 1999. HLA class I antigen downregulation in human cancers: T-cell immunotherapy revives an old story. Mol. Med. Today 5:178.
12. Seliger, B., M. J. Maeurer, and S. Ferrone. 2000. Antigen-processing machinery breakdown and tumor growth. Immunology Today 21:455.
13. Marincola, F. M., E. M. Jaffee, D. J. Hicklin, and S. Ferrone. 2000. Escape of human solid tumors from T-cell recognition: molecular mechanisms and functional significance. Adv. Immunology 74:181.
14. Khong, H. T., and N. P. Restifo. 2002. Natural selection of tumor variants in the generation of “tumor escape” phenotypes. Nat Immunol 3:999.
15. Dunn, G. P., A. T. Bruce, H. Ikeda, L. J. Old, and R. D. Schreiber. 2002. Cancer immunoediting: from immunosurveillance to tumor escape. Nature Immunol. 3:991.
16. Gromme, M., and J. J. Neefjes. 2002. Antigen degradation or presentation by MHC class I molecules via classical and non-classical pathways. Mol. Immunol. 39:181.
17. Seliger, B., M. J. Maeurer, and S. Ferrone. 1997. TAP off-Tumors on. Immunology Today 18:292.
18. Hill, A., P. Jugovic, I. York, G. Russ, J. R. Bennink, J. Yewdell, H. Ploegh, and D. Johnson. 1995. Herpes simplex virus turns off the TAP to evade host immunity. Nature 375:411.
19. Ahn, K., A. Gruhler, B. Galocha, T. R. Jones, E. J. Wiertz, H. L. Ploegh, P. A. Peterson, Y. Yang, and K. Fruh. 1997. The ER-luminal domain of the HCMV glycoprotein US6 inhibits peptide translocation by TAP. Immunity 6:613.
20. Boname, J. M., B. De Lima, P. J. Lehner, and P. G. Stevenson. 2004. Viral degradation of the MHC class I peptide loading complex. Immunity 20:305.
21. Koppers-Lalic, D., E. A. Reits, M. E. Ressing, A. D. Lipinska, R. Abele, J. Koch, M. Marcondes Rezende, P. Admiraal, D. Van Leeuwen, K. Bienkowska-Szewczyk, T. C. Mettenleiter, F. A. Rijsewijk, R. Tampe, J. J. Neefjes, and E. J. Wiertz. 2005. Varicelloviruses avoid T cell recognition by UL49.5-mediated inactivation of the transporter associated with antigen processing. Proc Natl Acad Sci USA 102:5144.
22. Fruh, K., K. Ahn, H. Djaballah, P. Sempe, P. Van Endert, R. Tampe, P. A. Peterson, and Y. Yang. 1995. A viral inhibitor of peptide transporters for antigen presentation. Nature 375:415.
23. Hengel, H., J. O. Koopmann, T. Flohr, W. Muranyi, E. Goulmy, G. J. Hammerling, U. H. Koszinowski, and F. Momburg. 1997. A viral ER-resident glycoprotein inactivates the MHC-encoded peptide transporter. Immunity 6:623.
24. Van Hall, T., E. Z. Wolpert, P. Van Veelen, S. Laban, M. Van der Veer, M. Roseboom, S. Bres, P. Grufman, A. De Ru, H. Meiring, A. De Jong, K. Franken, A. Teixeira, R. Valentijn, J. W. Drijfhout, F. Koning, M. Camps, F. Ossendorp, K. Karre, H. G. Ljunggren, C. J. Melief, and R. Offring a. 2006. Selective cytotoxic T-lymphocyte targeting of tumor immune escape variants. Nat Med 12:417.
25. Heemskerk M H, de Paus R A, Lurvink E G et al. Dual HLA class I and class II restricted recognition of alloreactive T lymphocytes mediated by a single T cell receptor complex. Proc. Natl. Acad. Sci. U.S.A 2001; 98:6806-11.
26. Mutis T, Verdijk R, Schrama E et al. Feasibility of immunotherapy of relapsed leukemia with ex vivo-generated cytotoxic T lymphocytes specific for hematopoietic system-restricted minor histocompatibility antigens. Blood 1999; 93:2336-41.
27. den Haan J M, Meadows L M, Wang W et al. The minor histocompatibility antigen HA-1: a diallelic gene with a single amino acid polymorphism. Science 1998; 279:1054-7.
28. den Haan J M, Sherman N E, Blokland E et al. Identification of a graft versus host disease-associated human minor histocompatibility antigen. Science 1995; 268:1476-80.
29. Wang W, Meadows L R, den Haan J M et al. Human H-Y: a male-specific histocompatibility antigen derived from the SMCY protein. Science 1995; 269:1588-90.
30. Mulder A, Kardol M, Regan J et al. Reactivity of twenty-two cytotoxic human monoclonal HLA antibodies towards soluble HLA class I in an enzyme-linked immunosorbent assay (PRA-STAT). Hum. Immunol. 1997; 56:106-13.
31. Momburg F, Ortiz-Navarrete V, Neefjes J et al. Proteasome subunits encoded by the major histocompatibility complex are not essential for antigen presentation. Nature 1992; 360:174-7.
32. Grandea A G, III, Lehner P J, Cresswell P et al. Regulation of MHC class I heterodimer stability and interaction with TAP by tapasin. Immunogenetics 1997; 46:477-83.
33. Lewis J W, Sewell A, Price D et al. HLA-A*0201 presents TAP-dependent peptide epitopes to cytotoxic T lymphocytes in the absence of tapasin. Eur. J. Immunol. 1998; 28:3214-20.
34. Corbett, T. H., D. P. Griswold, B. J. Roberts, J. C. Peckham, and F. M. Schable. 1975. Tumor induction relationships in development of transplantable cancers of the colon in mice for chemotherapy assays, with a note on carcinogen structure. Canc. Res. 35:2434.
35. Huang, A. Y. C., P. H. Gulden, A. S. Woods, M. C. Thomas, C. D. Tong, W. Wang, V. H. Engelhard, G. Pasternack, R. Cotter, D. Hunt, D. M. Pardoll, and E. M. Jaffee. 1996. The immunodominant major histocompatibility complex class I-restricted antigen of a murine colon tumor derives from an endogenous retroviral gene product. Proc. Natl. Aca. Sci. USA 93:9730.
36. Cotterill, L. A., H. J. Stauss, M. M. Millrain, D. J. Pappin, D. Rahman, B. Canas, P. Chandler, A. Stackpoole, P. J. Simpson, and P. J. Dyson. 1997. Qa-1 interaction and T cell recognition of the Qa-1 determinant modifier peptide. Eur. J. Immunol. 27:2123.
37. Van Hall, T., J. Van Bergen, P. Van Veelen, M. Kraakman, L. C. Heukamp, F. Koning, C. J. M. Melief, F. Ossendorp, and R. Offring a. 2000. Identification of a novel tumor-specific CTL epitope presented by RMA, EL-4, and MBL-2 lymphomas reveals their common origin. J. Immunol. 165:869.
38. Hermel, E., A. J. Hart, I. Gunduz, H. Acton, C. Kim, M. Wurth, S. Uddin, C. Smith, K. Fischer Lindahl, and C. J. Aldrich. 2004. Polymorphism and conservation of the genes encoding Qa1 molecules. Immunogenetics 56:639.
39. Schuurhuis, D. H., A. Ioan-Facsinay, B. Nagelkerken, J. J. Van Schip, C. Sedlik, C. J. Melief, J. S. Verbeek, and F. Ossendorp. 2002. Antigen-antibody immune complexes empower dendritic cells to efficiently prime specific CD8+ CTL responses in vivo. J. Immunol. 168:2240-2246.
40. Koppers-Lalic, D., Rychlowski, M., van Leeuwen, D., Rijsewijk, F. A., Ressing, M. E., Neefjes, J. J., Bienkowska-Szewczyk, K. & Wiertz, E. J. (2003) Arch. Virol. 148, 2023-2037.
41. Koppers-Lalic, D., Rijsewijk, F. A., Verschuren, S. B., van Gaans-Van den Brink, J. A., Neisig, A., Ressing, M. E., Neefjes, J. & Wiertz, E. J. (2001) J. Gen. Virol. 82, 2071-2081.
42. Anderson, W F. Human gene therapy. Nature (1998) 392: 25-30.
43. Walther W, Stein U. Viral vectors for gene transfer—A review of their use in the treatment of human diseases. Drugs (2000) 60: 249-71.
44. Kay M A, Glorioso J C, Naldini L. Viral vectors for gene therapy: the art of turning infectious agents into vehicles of therapeutics. Nat Med (2001) 7: 33-40.
45. Boczkowski, D., Nair, S. K., Snyder, D., and Gilboa, E. Dendritic cells pulsed with RNA are potent antigen-presenting cells in vitro and in vivo. J. Exp. Med., 184: 465-472, 1996.
46. Boczkowski, D., Nair, S. K., Nam, J.-H., Lyerly, H. K., and Gilboa, E., Induction of Tumor Immunity and Cytotoxic T Lymphocyte Responses Using Dendritic Cells Transfected with Messenger RNA Amplified from Tumor Cells, Cancer Res. (2000) 60, 1028-1034.
47. Coughlin, C. M., Vance, B. A., Grupp, S. A., and Vonderheide, R. H., RNA-transfected CD40-activated B cells induce functional T-cell responses against viral and tumor antigen targets: implications for pediatric immunotherapy, Blood (2004) 103: 2046-2054.
48. Strobl H., E. Riedl, C. Scheinecker, C. Bello-Fernandez, W. F. Pickl, K. Rappersberger, O. Majdic and W. Knapp. 1996. TGF-beta1 promotes in vitro development of dendritic cells from CD34+ hemopoietic progenitors. J. Immunology. 157(4):1499-1507.
49. Kiertscher, S M., and Roth M. D. 1996. Human C D 14+ leukocytes acquire the phenotype and function of antigen-presenting dendritic cells when cultured in GM-CSF and IL-4. Journal of Leukocyte Biology 59(2):208-218.
50. Coligan J E, Kruisbeek A M, Margulies D H, Schevach E M, Strober W. Preparation of human mononuclear cell populations and subpopulations. In: Coico R, ed. Current protocols in immunology. Vol. 2: John Wiley & Sons, Inc., 1994:711-94.

1. An in vitro method for producing a cell that induces CD8⁺ T lymphocytes that selectively recognize cells presenting T cell epitopes associated with impaired peptide processing (TEIPP), the method comprising treating the cell with an effective amount of a varicellovirus TAP-inhibitor, which has the following properties:
a) is a protein with at least 50% amino acid sequence identity with at least one of SEQ ID NO:1, SEQ ID NO:2, SEQ ID NO:3 and SEQ ID NO:4 or is a nucleic acid encoding said protein; and,
b) the TAP inhibitor protein reduces by at least 50% TAP-dependent transport of fluorescein-conjugated synthetic peptide CVNKTERAY (SEQ ID NO:14) into cells of human melanoma MEL-JUSO cell line that stably express the TAP-inhibitor as compared to TAP-dependent transport of the fluorescein-conjugated peptide into human melanoma MEL-JUSO cells that do not express the TAP-inhibitor.

2. The method according to claim 1, wherein the varicellovirus TAP-inhibitor protein is modified by deletion or replacement of at least one lysine, serine, threonine or cysteine residue in the cytoplasmic tail of the TAP-inhibitor protein, wherein replacement is with an amino acid residue other than lysine, serine, threonine and cysteine.

3. The method according to claim 2, wherein the cytoplasmic tail lacks lysine, serine, threonine and cysteine residues.

4. The method according to claim 1, wherein the varicellovirus TAP-inhibitor is said encoding nucleic acid molecule.

5. The method according to claim 4, wherein the nucleic acid molecule is:
a) an expression construct for transient expression in which the sequence encoding the TAP-inhibitor protein is operably linked to a promoter; or,
b) an RNA molecule.

6. The method according to claim 4, wherein the nucleotide sequence encoding the TAP-inhibitor protein exhibits at least one of the following properties:
a) a codon adaptation index for a human host cell of at least 0.3; and,
b) at least 50% of non-common codons or less-common codons are replaced with common codons encoding the same amino acid as shown in Table 3.

7. The method according to claim 4, wherein the nucleotide sequence encoding the TAP-inhibitor protein encodes the amino acid sequence SEQ ID NO:1 or SEQ ID NO:5, and has at least 60% nucleotide sequence identity with SEQ ID NO:10.

8. The method according to claim 1, wherein the cell being produced is an antigen presenting cell.

9. The method according to claim 8, wherein the antigen presenting cell is autologous to a subject in whom the cell is to be used as a therapeutic agent.

10. A method of treating cancer or a virus infection in a subject comprising administering to a subject in need thereof cells produced by a method according to claim 1.

11. The method according claim 10, wherein the cells activate CD8⁺ T lymphocytes that selectively recognize tumor cells or virus infected cells which present TEIPP.

12. A pharmaceutical composition comprising the cell according to claim 1 and a pharmaceutically acceptable carrier or excipient.

13. A nucleic acid molecule comprising a nucleotide sequence encoding a varicellovirus TAP-inhibitor protein that:
(a) has at least 50% amino acid identity with at least one of SEQ ID NO 1, SEQ ID NO:2, SEQ ID NO:3 and SEQ ID NO:4; and,
(b) reduces by at least 50% TAP-dependent transport of fluorescein-conjugated synthetic peptide CVNKTERAY (SEQ ID NO:14) into cells of human melanoma MEL-JUSO cell line that stably express the TAP-inhibitor as compared to TAP-dependent transport of the fluorescein-conjugated peptide into human melanoma MEL-JUSO cells that do not express the TAP-inhibitor.

14. The nucleic acid molecule according to claim 13, wherein the varicellovirus TAP-inhibitor protein
(a) lacks a lysine, serine, threonine or cysteine residue, or
(b) is modified by deletion or replacement of at least one lysine, serine, threonine or cysteine residues in the cytoplasmic tail of the TAP-inhibitor protein, wherein replacement is with an amino acid residue other than lysine, serine, threonine and cysteine.

15. (canceled)

16. The nucleic acid molecule according to claim 13, which is:
(a) an expression construct for transient expression in which the sequence encoding the TAP-inhibitor protein is operably linked to a promoter; or,
(b) an RNA molecule.

17. The nucleic acid molecule according claim 13, wherein the nucleotide sequence encoding the TAP-inhibitor protein exhibits at least one of the following properties:
(a) a codon adaptation index for a human host cell of at least 0.3; and,
(b) at least 50% of non-common codons or less-common codons are replaced with common codons encoding the same amino acid as shown in Table 3.

18. The nucleic acid molecule according to claim 13, wherein the sequence encoding the TAP-inhibitor protein encodes the amino acid sequence SEQ ID NO:1 or SEQ ID NO:5, and has at least 60% nucleotide sequence identity with SEQ ID NO:10.

19. (canceled)

20. A method for modifying a cell to:
(i) display an empty MHC class I molecules at its surface, or
(ii) to reduce surface expression of MHC class I molecules,
comprising treating the cell with a varicellovirus TAP-inhibitor protein or a nucleic acid molecule encoding said protein, thereby causing display of said empty MHC class I molecules or reducing said surface expression of MHC class I molecules.

21. (canceled)

22. A modified varicellovirus TAP-inhibitor, protein that has the following properties:
(a) at least 50% amino acid sequence identity with at least one of SEQ ID NO:1, SEQ ID NO:2, SEQ ID NO:3 and SEQ ID NO:4;
(b) reduces by at least 50% TAP-dependent transport of fluorescein-conjugated synthetic peptide CVNKTERAY (SEQ ID NO:14) into cells of human melanoma MEL-JUSO cell line that stably express the TAP-inhibitor, as compared to TAP-dependent transport of the fluorescein-conjugated peptide into human melanoma MEL-JUSO cells that do not express the TAP-inhibitor; and,
(c) lacks lysine, serine, threonine and cysteine residues in its cytoplasmic tail, or is modified by deletion or replacement of at least one lysine, serine, threonine or cysteine residues in its cytoplasmic tail, wherein replacement is with an amino acid residue other than lysine, serine, threonine and cysteine.

23. (canceled)

24. The method according to claim 8 wherein the antigen-presenting cell is a dendritic cell.

25. The method according to claim 11 wherein the TEIPP is MHC class I dependent.


SEQUENCE LISTING (13 sequences)

SEQ ID NO: 1 - protein, 75 aa, Bovine herpesvirus 1
RDPLLDAMRREGAMDFWSAGCYARGVPLSEPPQALVVFYVALTAVMVAVALYAYGLCFRLMGASGPNKKESRGRG

SEQ ID NO: 2 - protein, 73 aa, Pseudorabies virus
IVSTEGPLPLLREESRINFWNAACAARGVPVDQPTAAAVTFYICLLAVLVVALGYATRTCTRMLHASPAGRRV

SEQ ID NO: 3 - protein, 73 aa, Equid Herpesvirus 1
DPGVKQRIDVAREEERRDFWHAACSGHGFPITTPSTAAILFYVSLLAVGVAVACQAYRAVLRIVTLEMLQHLH

SEQ ID NO: 4 - protein, 74 aa, Equid Herpesvirus 4
GDLEAKQRLDVAREEERRDFWHAACSGHGFPITTPSTAAILFYVSLLAVGVAVACQAYRAFLRIVTLEMLRHLH

SEQ ID NO: 5 - protein, 96 aa, Bovine herpesvirus 1
MPRSPLIVAVVAAALFAIVRGRDPLLDAMRREGAMDFWSAGCYARGVPLSEPPQALVVFYVALTAVMVAVALYAYGLCFRLMGASGPNKKESRGRG

SEQ ID NO: 6 - protein, 98 aa, Pseudorabies virus
MVSSAGLSLTLVAALCALVAPALSSIVSTEGPLPLLREESRINFWNAACAARGVPVDQPTAAAVTFYICLLAVLVVALGYATRTCTRMLHASPAGRRV

SEQ ID NO: 7 - protein, 100 aa, Equid Herpesvirus 1
MLSTRFVTLAILACLLVVLGLARGAGGDPGVKQRIDVAREEERRDFWHAACSGHGFPITTPSTAAILFYVSLLAVGVAVACQAYRAVLRIVTLEMLQHLH

SEQ ID NO: 8 - protein, 100 aa, Equid Herpesvirus 4
MLSARLVTLAILTCLLVVFGLTRGASGDLEAKQRLDVAREEERRDFWHAACSGHGFPITTPSTAAILFYVSLLAVGVAVACQAYRAFLRIVTLEMLRHLH

SEQ ID NO: 9 - DNA, 291 bp, Bovine herpesvirus 1
atgccgcggtcgccgctcatcgttgcggttgtggccgccgcgctgtttgccatcgtgcgcggccgcgaccccctgctagacgcgatgcggcgcgagggggcaatggacttttggagcgcaggctgctacgcgcgcggggtgccgctctcggagccaccgcaggccctggttgttttttacgtggccctgaccgcggtaatggtcgccgtggccctgtacgcgtacgggctttgctttaggctcatgggcgccagcgggcccaataaaaaggagtcgcgggggcggggctga

SEQ ID NO: 10 - DNA, 291 bp, Bovine herpesvirus 1
atgcccagatcccccctgatcgtggccgtggtggccgccgccctgttcgccatcgtgcggggcagggaccccctgctggacgccatgcggcgggagggcgccatggacttttggagcgccggctgctacgccagaggcgtgcccctgagcgagccccctcaggccctggtggtgttctacgtggccctgaccgccgtgatggtggccgtggccctgtacgcctacggcctgtgcttccggctgatgggcgccagcggccccaacaagaaagagagccggggcaggggctga

SEQ ID NO: 11 - protein, 88 aa, Herpes Simplex Virus 1
MSWALEMADTFLDTMRVGPRTYADVRDEINKRGREDREAARTAVHDPERPLLRSPGLLPEIAPNASLGVAHRRTGGTVTDSPRNPVTR

SEQ ID NO: 12 - protein, 86 aa, Herpes Simplex Virus 2
MSWALKTTDMFLDSSRCTHRTYGDVCAEIHKREREDREAARTAVTDPELPLLCPPDVRSDPASRNPTQQTRGCARSNERQDRVLAP

SEQ ID NO: 13 - protein, 183 aa, Cytomegalovirus
MDLLIRLGFLLMCALPTPGERSSRDPITLLSLSPRQQACVPRTKSYRPVCYNDTGDCTDADDSWKQLSEDFAHQCLQAAKKRPKTHKSRPNDRNLEGRLTCQRVSRLLPCDLDIHPSHRLLTLMNDCVCDGAVWNAFRLIERHGFFAVTLYLCCGITLLVVILALLCSITYESTGRGIRRCGS